(19) United States
(12) Patent Application Publication
(10) Pub. No.: US 2025/0258159 A1
(43) Pub. Date: Aug. 14, 2025

(54) DEVICE, METHOD AND SYSTEM FOR MONITORING IMMUNE SYSTEM RESPONSE

(71) Applicant: Olaris, Inc., Framingham, MA (US)

(72) Inventors: Srihari Raghavendra Rao, Waltham, MA (US); Elizabeth M. O'Day, Franklin, MA (US); Leonardo O. Rodrigues, Framingham, MA (US); Chandrashekhar Honrao, Arlington, MA (US)

(21) Appl. No.: 19/188,733

(22) Filed: Apr. 24, 2025

Related U.S. Application Data
(62) Division of application No. 17/668,108, filed on Feb. 9, 2022.
(60) Provisional application No. 63/154,993, filed on Mar. 1, 2021.

(51) Int. Cl. G01N 33/50; G01N 21/25; G16H 20/10; G16H 50/30; G16H 50/70

(52) U.S. Cl. CPC G01N 33/5008; G01N 21/25; G16H 20/10; G16H 50/30; G16H 50/70

(57) ABSTRACT

The invention relates generally to monitoring the immune system response, and more specifically to identifying and diagnosing patients who are at risk for developing a disease or disorder associated with altered immune system response or who have been treated with a drug such as an immunosuppressant and using metabolomics and making a comparison against a database of patients to determine if an effective immune response has been elicited.

### CROSS-REFERENCE TO RELATED APPLICATIONS

This application is a divisional of U.S. application Ser. No. 17/668,108, filed Feb. 9, 2022, which claims benefit under 35 U.S.C. § 119(e) of provisional application 63/154,993, filed Mar. 1, 2021, which applications are hereby incorporated by reference in their entireties.

### FIELD OF THE INVENTION

The invention relates generally to the field of metabolite analysis and more particularly to spectrographic analysis of metabolites to provide information to patients and caregivers to improve treatment protocols.

### BACKGROUND OF THE INVENTION

The immune system has evolved to be able to detect and eliminate foreign pathogens, and damaged or diseased tissues and organelles. The system mounts a carefully orchestrated response to distinguish nefarious and benign entities (ie non-self vs self). Thus, the immune system response (ISR) plays a crucial role in maintaining human health, fighting disease, and determining the efficacy of therapeutic interventions. Altered ISR, either an over-active response or weakened response, can have severe consequences, such as the development of autoimmune diseases, the inability to fight infections, malignancy development, and drug treatment failures. Organ transplant provides an example of where altered ISR can lead to graft rejection, infection, malignancy, graft dysfunction and/or graft failure, and even death. For example, transplant recipients are usually administered an immunosuppressant, to dampen the ISR. Patients receiving subtherapeutic dosages of immunosuppressants may become under-immunosuppressed. Underimmunosuppression can lead to the formation of donor-specific antibodies (DSAs) and graft loss due to anti-body-mediated rejection (AMR). Too high of an immunosuppressant dose and patients may become over-immunosuppressed. This puts transplant patients at risk for infections and malignancy. Specific to kidney transplant patients, over-immunosuppression can lead to reactivation of the polyomavirus BK virus (BKV) and BKV-associated interstitial nephritis (BKVIN), leading to damage of the graft, and ultimately graft loss. It is a challenge for clinicians to find the delicate balance between under- and over-immunosuppression for transplant patients. The device described herein is able to monitor the ISR, making it possible to diagnose and identify patients at risk for disease and disorders associated with altered ISR.

### SUMMARY OF THE INVENTION

An aspect of the invention is a method of determining if a patient has an altered (either over-active or weakened) immune system response (ISR), comprising:
a. obtaining a biological sample from the patient; and
b. analyzing metabolites in a biological sample from the patient.

In another aspect of the invention the biological sample is selected from the group consisting of blood, urine, feces, cerebral fluid, saliva and tissue extract; wherein the analyzing comprises scanning the biological sample using spectroscopy to obtain data related to metabolites, and wherein the analyzing further comprises relating the data to data obtained from a statistically significant group of samples from patients previously analyzed.

In another aspect of the invention the statistically significant group of samples comprises samples from patients known to have an altered ISR and patients known to have a normal ISR, and wherein the analyzing comprises scanning the biological sample using nuclear magnetic resonance (NMR) spectroscopy and mass spectrometry.

In another aspect of the invention the sample is urine, and the NMR spectroscopy is two-dimensional NMR spectroscopy.

In another aspect of the invention the sample is urine, and the NMR spectroscopy is ¹H-¹³C heteronuclear single quantum correlation (HSQC) two-dimensional NMR spectroscopy.

In another aspect of the invention the sample is urine, and the NMR spectroscopy is one-dimensional NMR spectroscopy.

In another aspect of the invention the sample is urine, and the spectroscopy is mass spectrometry.

The invention includes analyzing the immune system response of a patient to make it possible to treat patients more effectively. The method includes first obtaining a biological sample from the patient which sample may be urine and then subjecting the sample to analysis such as by spectroscopy where the analysis is focused particularly on metabolites in the sample related to the patient's immune system response. The analysis of the sample is compared against a large database of information created from a statistically significant group of samples. Comparisons are carried out to determine a differential between results obtained with the patient and known results in the database. The comparison makes it possible to determine the overall health of the patient's immune system relative to a large sample of patients who have both healthy and unhealthy immune systems.

The invention comprises analyzing an immune system response in a kidney transplant patient to make it possible to improve patient outcomes. A urine sample is obtained from the patient and subjected to spectroscopy analysis using heteronuclear single-quantum correlation (HSQC) spectroscopy focused particularly on metabolites related to the patient's immune system response. The analysis of the sample is compared against a large database of information created from a statistically significant group of samples from other kidney transplant patients. The comparisons are carried out in order to determine a differential between results obtained with the patient and known results in the database. The comparison makes it possible to determine the overall health of the patient's immune system relative to a large sample of patients who have both healthy and unhealthy immune systems.

These and other objects, advantages, and features of the invention will become apparent to those persons skilled in the art upon reading the details of the methods, uses, and procedures as more fully described below.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 displays levels measured by NMR spectroscopy of metabolite resonances at 1.991+/−0.25 ppm×40.132+/−0.45 ppm (1A), 2.566+/−0.25 ppm×47.724+/−0.45 ppm (1B), 2.712+/−0.25 ppm×47.752+/−0.45 ppm (1C), 3.787+/−0.25 ppm×73.579+/−0.45 ppm (1D), 3.813+/−0.25 ppm×62.593+/−0.45 ppm (1E), 3.876+/−0.25 ppm×35.932+/−0.45 ppm (1F), 3.969+/−0.25 ppm×46.487+/−0.45 ppm (1G), 4.44+/−0.25 ppm×50.942+/−0.45 ppm (1H), 6.914+/−0.25 ppm×115.425+/−0.45 ppm (1I), 6.974+/−0.25 ppm×120.636+/−0.45 ppm (1J), 7.085+/−0.25 ppm×121.7+/−0.45 ppm (1K), 7.274+/−0.25 ppm×116.531+/−0.45 ppm (1L), 7.294+/−0.25 ppm×132.721+/−0.45 ppm (1M), 7.346+/−0.25 ppm×121.584+/−0.45 ppm (1N), 7.531+/−0.25 ppm×129.748+/−0.45 ppm (1O), 7.531+/−0.25 ppm×131.363+/−0.45 ppm (1P), 7.532+/−0.25 ppm×134.804+/−0.45 ppm (1Q), 7.618+/−0.25 ppm×134.808+/−0.45 ppm (1R), 7.624+/−0.25 ppm×131.123+/−0.45 ppm (1S), 7.817+/−0.25 ppm×131.402+/−0.45 ppm (1T), 7.818+/−0.25 ppm×129.762+/−0.45 ppm (1U), 8.08+/−0.25 ppm×130.252+/−0.45 ppm (1V), 8.841+/−0.25 ppm×147.236+/−0.45 ppm (1W), 9.117+/−0.25 ppm×148.323+/−0.45 ppm (1X) in the ¹H and ¹³C dimensions respectively that were significantly different between altered ISR patients and control patients.

FIG. 2 is the receiver operator curve (ROC) for the machine learning algorithm based on differential metabolites which produced a biomarker of response (BoR) score that differentiates altered ISR subjects from healthy controls with 81.1% cvAUC.

FIG. 3 displays metabolite levels measured by NMR spectroscopy for levels of metabolite resonances at 2.792+/−0.25 ppm×40.011+/−0.45 ppm (3A), 3.714+/−0.25 ppm×72.206+/−0.45 ppm (3B), 3.009+/−0.25 ppm×32.551+/−0.45 ppm (3C) in the ¹H and ¹³C dimensions respectively that were significantly different in kidney transplant subjects who were under-immunosuppressed compared to control subjects.

FIG. 4 is the receiver operator curve (ROC) for the machine learning algorithm based on differential metabolites which produce a biomarker of response (BoR) score that differentiates kidney transplant subjects who were under-immunosuppressed from controls with 87.1% cvAUC.

FIG. 5 displays metabolite levels measured by NMR spectroscopy for levels of metabolite resonances at 2.512+/−0.25 ppm×28.032+/−0.45 ppm (5A), 2.787+/−0.25 ppm×40.035+/−0.45 ppm (5B), 3.01+/−0.25 ppm×32.525+/−0.45 ppm (5C), 3.637+/−0.25 ppm×78.911+/−0.45 ppm (5D), 3.714+/−0.25 ppm×72.229+/−0.45 ppm (5E), 3.722+/−0.25 ppm×75.654+/−0.45 ppm (5F), 3.851+/−0.25 ppm×64.395+/−0.45 ppm (5G), 3.966+/−0.25 ppm×46.482+/−0.45 ppm (5H), 5.016+/−0.25 ppm×74.051+/−0.45 ppm (5I), 6.914+/−0.25 ppm×115.449+/−0.45 ppm (5J), 6.974+/−0.25 ppm×120.651+/−0.45 ppm (5K), 7.088+/−0.25 ppm×121.707+/−0.45 ppm (5L), 7.276+/−0.25 ppm×116.555+/−0.45 ppm (5M), 7.533+/−0.25 ppm×131.372+/−0.45 ppm (5N), 7.534+/−0.25 ppm×129.772+/−0.45 ppm (5O), 7.534+/−0.25 ppm×134.842+/−0.45 ppm (5P), 7.619+/−0.25 ppm×134.811+/−0.45 ppm (5Q), 7.817+/−0.25 ppm×131.398+/−0.45 ppm (5R) in the ¹H and ¹³C dimensions respectively that were significantly different between under and over-immunosuppressed subjects.

FIG. 6 is the receiver operator curve (ROC) for the machine learning algorithm based on differential metabolites which produce a biomarker of response (BoR) score that differentiates between under and over-immunosuppressed subjects with 90.9% cvAUC.

FIG. 7 displays metabolite levels measured by NMR spectroscopy for levels of metabolite resonances at 2.711+/−0.25 ppm×47.702+/−0.45 ppm (7A), 3.969+/−0.25 ppm×46.484+/−0.45 ppm (7B), 7.086+/−0.25 ppm×121.698+/−0.45 ppm (7C), 7.274+/−0.25 ppm×116.53+/−0.45 ppm (7D), 7.347+/−0.25 ppm×121.578+/−0.45 ppm (7E), 7.532+/−0.25 ppm×129.75+/−0.45 ppm (7F), 7.532+/−0.25 ppm×131.359+/−0.45 ppm (7G), 7.533+/−0.25 ppm×134.812+/−0.45 ppm (7H), 7.618+/−0.25 ppm×134.804+/−0.45 ppm (7I), 7.817+/−0.25 ppm×131.389+/−0.45 ppm (7J), 7.818+/−0.25 ppm×129.746+/−0.45 ppm (7K), 9.117+/−0.25 ppm×148.321+/−0.45 ppm (7L) in the ¹H and ¹³C dimensions respectively that were significantly different between subjects who had appropriate ISR compared to those that did not.

FIG. 8 is the receiver operator curve (ROC) for the machine learning algorithm based on differential metabolites which produce a biomarker of response (BoR) score that differentiates subjects who had appropriate ISR compared to those that did not with 75% cvAUC.

FIG. 9 displays metabolite levels measured by NMR spectroscopy for levels of metabolite resonances at 2.19+/−0.25 ppm×24.54+/−0.45 ppm (9A), 2.22+/−0.25 ppm×39.75+/−0.45 ppm (9B), 2.82+/−0.25 ppm×30+/−0.45 ppm (9C), 2.89+/−0.25 ppm×32.96+/−0.45 ppm (9D), 3.12+/−0.25 ppm×32.81+/−0.45 ppm (9E), 3.38+/−0.25 ppm×76.2+/−0.45 ppm (9F), 3.39+/−0.25 ppm×76.25+/−0.45 ppm (9G), 3.62+/−0.25 ppm×78.2+/−0.45 ppm (9H), 3.75+/−0.25 ppm×62+/−0.45 ppm (9I), 3.97+/−0.25 ppm×46.51+/−0.45 ppm (9J), 4.05+/−0.25 ppm×58.5+/−0.45 ppm (9K), 4.45+/−0.25 ppm×50.9+/−0.45 ppm (9L), 7.534+/−0.25 ppm×131.3+/−0.45 ppm (9M), 7.619+/−0.25 ppm×134.8+/−0.45 ppm (9N), 7.62+/−0.25 ppm×131.1+/−0.45 ppm (9O), 7.82+/−0.25 ppm×129.7+/−0.45 ppm (9P) in the ¹H and ¹³C dimensions respectively that were significantly different between subjects who developed BKVIN and control subjects.

FIG. 10 is the receiver operator curve (ROC) for the machine learning algorithm based on differential metabolites which produce a biomarker of response (BoR) score that differentiates subjects who developed BKVIN and control subjects with 91.5% cvAUC.

FIG. 11 displays metabolite levels measured by NMR spectroscopy for levels of metabolite resonances at 2.2+/−0.25 ppm×39.75+/−0.45 ppm (11A), 2.82+/−0.25 ppm×30+/−0.45 ppm (11B), 3.71+/−0.25 ppm×72.1+/−0.45 ppm (11C), 3.75+/−0.25 ppm×62+/−0.45 ppm (11D), 3.973+/−0.25 ppm×46.56+/−0.45 ppm (11E), 4.05+/−0.25 ppm×58.6+/−0.45 ppm (11F), 4.45+/−0.25 ppm×50.8+/−0.45 ppm (11G), 7.5+/−0.25 ppm×129.7+/−0.45 ppm (11H), 7.533+/−0.25 ppm×134.8+/−0.45 ppm (11I), 7.534+/−0.25 ppm×131.3+/−0.45 ppm (11J), 7.618+/−0.25 ppm×134.7+/−0.45 ppm (11K), 7.62+/−0.25 ppm×131.1+/−0.45 ppm (11L), 7.8+/−0.25 ppm×131.4+/−0.45 ppm (11M), 7.82+/−0.25 ppm×129.7+/−0.45 ppm (11N), 9.11+/−0.25 ppm×148.25+/−0.45 ppm (11O) in the ¹H and ¹³C dimensions respectively that were significantly different between male over-immunosuppressed subjects and male control subjects.

FIG. 12 is the receiver operator curve (ROC) for the machine learning algorithm based on differential metabolites which produce a biomarker of response (BoR) score that differentiates male over-immunosuppressed subjects and male control subjects with 100% cvAUC.

FIG. 13 displays metabolite levels measured by NMR spectroscopy for levels of metabolite resonances at 1.27+/−0.25 ppm×30.8+/−0.45 ppm (13A), 1.91+/−0.25 ppm×32.6+/−0.45 ppm (13B), 2.22+/−0.25 ppm×39.75+/−0.45 ppm (¹³C), 2.51+/−0.25 ppm×28.05+/−0.45 ppm (13D), 3.12+/−0.25 ppm×32.76+/−0.45 ppm (13E), 3.163+/−0.25 ppm×44.09+/−0.45 ppm (13F), 3.25+/−0.25 ppm×30.33+/−0.45 ppm (13G), 3.38+/−0.25 ppm×76.2+/−0.45 ppm (13H), 3.6+/−0.25 ppm×73.11+/−0.45 ppm (131), 3.72+/−0.25 ppm×69.98+/−0.45 ppm (13J), 7.16+/−0.25 ppm×122.3+/−0.45 ppm (13K), 7.48+/−0.25 ppm×114.6+/−0.45 ppm (13L), 7.65+/−0.25 ppm×119.8+/−0.45 ppm (13M) in the ¹H and ¹³C dimensions respectively that were significantly different between female over-immunosuppressed subjects and female control subjects.

FIG. 14 is the receiver operator curve (ROC) for the machine learning algorithm based on differential metabolites which produce a biomarker of response (BoR) score that differentiates female over-immunosuppressed subjects and female control subjects with 100% cvAUC.

### DETAILED DESCRIPTION OF THE INVENTION

Before the present methods, uses and procedures are described, it is to be understood that this invention is not limited to the particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described.

All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a sample” includes a plurality of such samples and reference to “the patient” includes reference to one or more patients and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

### Experimental Protocol

It is to be understood that the invention is not limited to the particular methodologies, protocols, cell lines, assays, and reagents described herein as they may vary. It is also to be understood that the terminology used herein is intended to describe particular embodiments of the present invention and is in no way intended to limit the scope of the present invention as set forth in the appended claims.

The present invention is based, in part, on the discovery of unexpected changes (increases and decreases) in certain metabolite biomarkers in the urine of subjects having altered immune system response (e.g. kidney transplant patients who develop BK virus interstitial nephritis (BKVIN)). The present invention demonstrates that these metabolite levels may be assayed to diagnose altered (either over-active or weakened) immune response in a subject. The present invention further shows that measurements of certain biomarkers in the urine from a subject may be used to predict the subsequent development and progression of a disease due to the altered immune response (e.g. identify a kidney transplant subject at risk of developing BKVIN and/or identify a subject with a progression of immune disorder such as graft loss, rejection, or dysfunction in transplant patients, infection, or malignancy).

The present invention also provides compositions of use in the methods described herein. Such compositions may include endogenous metabolites, microbiome byproducts, and xenobiotics. The present invention further provides kits for diagnosing or prognosing an altered immune system response (ISR) in a subject, identifying a subject at risk of a disease or disorder due to altered ISR or prescribing a therapeutic regimen or predicting benefit from therapy in a subject having altered ISR. The section headings are used herein for organizational purposes only and are not to be construed as in any way limiting the subject matter described herein.

### Biological Sample

The present invention provides biomarkers and diagnostic and prognostic methods for altered immune system response (ISR) and other diseases or disorders that result from altered ISR. Biomarker levels are determined in a biological sample obtained from a subject. In some embodiments, the biological sample of the invention can be obtained from blood. Blood may be combined with various components following collection to preserve or prepare samples for subsequent techniques. For example, in some embodiments, blood is treated with an anticoagulant, a cell fixative, a protease inhibitor, a phosphatase inhibitor, a protein, a DNA, or an RNA preservative following collection. In some embodiments, blood is collected via venipuncture using vacuum collection tubes containing an anticoagulant such as EDTA or heparin. Blood can also be collected using a heparin-coated syringe and hypodermic needle. Biological samples can also be obtained from other sources known in the art, including whole blood, serum, plasma, urine, interstitial fluid, peritoneal fluid, cervical swab, tears, saliva, buccal swab, skin, cerebrospinal fluid, or other tissues including for example brain tissues. Preservative methods specific to each biofluid may be used.

### Metabolite Extraction and Quantification

The present invention provides metabolite biomarkers and diagnostic and prognostic methods for altered immune system response (ISR) and other diseases or disorders that result from altered ISR. The metabolites are extracted from a biological sample obtained from a subject. In some embodiments the metabolites can be extracted from urine. Metabolites can also be extracted from other sources known in the art, including whole blood, serum, plasma, urine, interstitial fluid, peritoneal fluid, cervical swab, tears, saliva, buccal swab, skin, cerebrospinal fluid or other tissues including for example brain tissues. Metabolites can be extracted from a significant group of biological samples collected from patients known to have a disease or disorder associated with an altered immune system response and patients known to have a normal immune response.

In some embodiments, metabolites can be extracted using organic precipitation. In some embodiments, methanol, chloroform and centrifugation are used to precipitate proteins and macromolecules. In some embodiments, filtration can also be used to separate and isolate metabolites. In some embodiments the solution enriched with metabolites may be dried and metabolites resuspended in a different solvent. In some embodiments, metabolite levels are measured using NMR spectroscopy including 1D and 2D methods. In some embodiments metabolite levels are measured using mass spectrometry (MS).

Diseases or Disorders with Altered Immune System Response

The present invention provides methods for diagnosing or prognosing altered immune system response (ISR) in a subject, identifying a subject at risk of a disease or disorder related to altered ISR, identifying a subject at risk of an infection, disease, or disorder that results from altered ISR, or prescribing a therapeutic regimen or predicting benefit from therapy in a subject with altered ISR or at risk of an infection, disease, or disorder that results from altered ISR.

In some embodiments, the disease or disorder related to or resulting from altered ISR is selected from the group consisting of: Infectious and inflammatory disorders, allergic and autoimmune diseases, Insulin-dependent diabetes mellitus, rheumatoid arthritis (RA), psoriasis, psoriatic arthritis, multiple sclerosis (MS), systemic lupus erythematosus (SLE), inflammatory bowel disease, Addison's disease, Grave's disease, Sjören's syndrome, Hasimoto's thyroiditis, Myasthenia gravis, Autoimmune vasculitis, Pernicious anemia, Celiac disease, thyrotoxicosis, autoimmune atrophic gastritis, Goodpasture syndrome, sympathetic ophthalmia, autoimmune hemolytic anemia, ulcerative colitis, scleroderma, Chron's disease, primary biliary cirrhosis, Guillain-Barre syndrome, ankylosing spondylitis, glucocorticoid-responsive conditions, acute asthma, giant cell arteritis, idiopathic thrombocytopenia purpura, advanced pulmonary/extrapulmonary tuberculosis, autoimmune hepatitis, Chron's disease, shock, lowering of hypercalcemia, promotion of water excretion, treatment of pathologic hypoglycemia, suppression of excess adrenocortical secretion, prevention of graft rejection, neurological disorders, hematological disorders, cardiovascular disorders, skin disorders, malignancies, and corticosteroid replacement therapy.

In some embodiments, the disease or disorder that patients with altered ISR are at risk is selected from the group consisting of: infectious and inflammatory disorders, viral infections, bacterial infections, fungal infections, Polyoma virus-associated nephropathy (PVAN), BK virus infection, BK viruria, BK viremia, BK virus interstitial nephritis (BKVIN), JC virus associated progressive multifocal leukoencephalopathy (PML), cytomegalovirus infections, CMV viremia, CMV disease, urinary tract infections (UTI), varicella zoster infection, herpes simplex infection, Epstein-Barr Virus (mononucleosis) infection, allergic and autoimmune diseases, Graft-versus-host-rejection disorder (GVHD), neurological disorders, hematological disorders, cardiovascular disorders, skin disorders, malignancies, new primary malignancy, skin cancer, lymphoma, graft dysfunction, graft failure, graft rejection, and post-transplant lymphoproliferative disorder.

In some embodiments, the disease or disorder that patients with altered ISR requiring immunosuppressant such as tacrolimus, cyclosporin A, calcineurin inhibitors, corticosteroids, mycophenolate mofetil (MMF), induction therapy in all formulations, including but not limited to, oral solid formulations, oral liquid formulations, extemporaneous compounding-oral formulations, injectable administration, intravenous administration, and topical administration, are selected from the group consisting of: Liver transplant rejection prophylaxis, kidney transplant rejection prophylaxis, heart transplant rejection prophylaxis, atopic dermatitis, acute liver transplant rejection, pancreas transplant rejection prophylaxis, islet transplantation rejection prophylaxis, small bowel transplant rejection prophylaxis, graft-versus-host disease (GVHD), chronic allergic contact dermatitis, psoriasis, facial or intertriginous psoriasis, seer, refractory uveitis, steroid- and cyclosporin-resistant nephrotic syndrome, steroid-refractory moderately to severely active ulcerative colitis, vulvar lichen sclerosis, lupus nephritis, kidney transplant rejection recipients with elected unresectable or metastatic cancers, bone marrow transplant in patients with severe sickle cell, bone marrow transplant in patients with high-risk solid tumors, prevention of GVHD in patients with acute leukemia, myelodysplastic syndrome, myelofibrosis undergoing reduced intensity conditioning donor stem cell transplantation, superficial kaposiform hemangioendothelioma and tufted angioma, refractory vernal keratoconjunctivitis, atopic keratoconjunctivitis, refractory nephrotic syndrome, pediatric heart transplant, minimal change disease (kidney), HBV associated glomerulonephritis, refractory pure red cell aplasia, refractory autoimmune cytopenia, dry eye, hereditary hemorrhagic telangiectasia, and adult facial seborrheic dermatitis.

In some embodiments, the type of solid organ transplant, in adults or in pediatrics, related to the prevention of graft failure, graft rejection, treatment of graft dysfunction, or treatment of acute graft rejection in patients with altered ISR is selected from a group consisting of: Kidney transplant, heart transplant, intestinal (small bowel) transplant, islet cell transplant, liver transplant, lung transplant, pancreas transplant, and bone marrow transplant.

In some embodiments, the present invention enables a medical practitioner to diagnose altered ISR and one or more diseases or disorders in a subject. In other embodiments, the present invention enables a medical practitioner to rule out or eliminate one or more diseases or disorders associated with altered ISR in a patient as a diagnostic possibility. In yet other embodiments, the present invention enables a medical practitioner to identify a subject at risk of developing a disease or disorder associated with altered ISR. In other embodiments, the present invention enables a medical practitioner to predict whether a subject will later develop a disease or disorder associated with altered ISR. In further embodiments the present invention enables a medical practitioner to prescribe a therapeutic regimen or predict benefit from therapy in a subject having altered ISR.

The present invention comprises a method of determining a point at which a patient develops a disease or disorder associated with altered immune system response, comprising (a) analyzing a metabolite in a human biological sample of a patient at a first point in time; analyzing the sample of the patient at a point in time different from the analyzing in step (a); comparing the analyzing of (a) with the analyzing of (b) to obtain a differential; and (d) relating the differential to a standard in order to determine if the patient has developed a disease or disorder associated with altered an altered immune response. In some embodiments, the present invention enables counseling the patient regarding developing a disease or disorder associated with altered-ISR; discontinuing administration of a drug to a patient who is at risk of a disease or disorder associated with altered-ISR, and adjusting the dose of a drug of a drug to a patient who is at risk of a disease or disorder associated with altered-ISR.

### Biomarkers

Biomarker levels are assayed in a biological sample obtained from a subject having or at-risk of having altered immune system response (ISR). In some embodiments the biomarker is choline, hippuric acid, indole-3-acetic acid, lysine, trigonelline, tryptophan, 2-pyrocatechuic-acid, 3-Hydroxymandelic acid,L-Phenylalanine,4-Methoxyphenylacetic acid, 4-Aminohippuric acid, Pteroyltriglutamic acid, 4-Ethylbenzoic acid, Aspartylphenylalanine, Creatinine, Diphenhydramine, D-Xylose, Gulonic acid, Hippuric acid, Homoveratric acid, Pyroglutamic acid, Quinic acid, Salicyluric acid, trigonelline (Table 1), and metabolite resonances detected via 2D ¹H-¹³C HSQC NMR spectroscopy at 1.275+/−0.25 ppm×30.8+/−0.45 ppm, 2.195+/−0.25 ppm×24.54+/−0.45 ppm, 2.22+/−0.25 ppm×39.75+/−0.45 ppm, 2.51+/−0.25 ppm×28.05+/−0.45 ppm, 2.825+/−0.25 ppm×30+/−0.45 ppm, 2.891+/−0.25 ppm×32.96+/−0.45 ppm, 3.119+/−0.25 ppm×32.76+/−0.45 ppm, 3.123+/−0.25 ppm×32.81+/−0.45 ppm, 3.166+/−0.25 ppm×44.04+/−0.45 ppm, 3.25+/−0.25 ppm×30.3+/−0.45 ppm, 3.38+/−0.25 ppm×76.2+/−0.45 ppm, 3.591+/−0.25 ppm×73.11+/−0.45 ppm, 3.62+/−0.25 ppm×78.2+/−0.45 ppm, 3.71+/−0.25 ppm×72.1+/−0.45 ppm, 3.717+/−0.25 ppm×69.98+/−0.45 ppm, 3.75+/−0.25 ppm×62+/−0.45 ppm, 3.9+/−0.25 ppm×79+/−0.45 ppm, 7.1+/−0.25 ppm×122+/−0.45 ppm, 7.533+/−0.25 ppm×129.7+/−0.45 ppm, 7.625+/−0.25 ppm×131.1+/−0.45 ppm, 7.65+/−0.25 ppm×119.85+/−0.45 ppm, 7.819+/−0.25 ppm×131.4+/−0.45 ppm, 3.876+/−0.25 ppm×35.932+/−0.45 ppm, 7.624+/−0.25 ppm×131.123+/−0.45 ppm, 7.532+/−0.25 ppm×134.804+/−0.45 ppm, 3.813+/−0.25 ppm×62.593+/−0.45 ppm, 1.991+/−0.25 ppm×40.132+/−0.45 ppm, 7.294+/−0.25 ppm×132.721+/−0.45 ppm, 4.44+/−0.25 ppm×50.942+/−0.45 ppm, 3.787+/−0.25 ppm×73.579+/−0.45 ppm, 6.914+/−0.25 ppm×115.425+/−0.45 ppm, 6.974+/−0.25 ppm×120.636+/−0.45 ppm, 7.085+/−0.25 ppm×121.7+/−0.45 ppm, 7.817+/−0.25 ppm×131. 402+/−0.45 ppm, 8.841+/−0.25 ppm×147.236+/−0.45 ppm, 3.969+/−0.25 ppm×46. 487+/−0.45 ppm, 8.08+/−0.25 ppm×130.252+/−0.45 ppm, 7.531+/−0.25 ppm×131. 363+/−0.45 ppm, 7.618+/−0.25 ppm×134.808+/−0.45 ppm, 7.818+/−0.25 ppm×129.762+/−0.45 ppm, 9.117+/−0.25 ppm×148. 323+/−0.45 ppm, 7.531+/−0.25 ppm×129.748+/−0.45 ppm, 7.346+/−0.25 ppm×121.584+/−0.45 ppm, 7.274+/−0.25 ppm×116.531+/−0.45 ppm, 2.566+/−0.25 ppm×47.724+/−0.45 ppm, 2.712+/−0.25 ppm×47.752+/−0.45 ppm, 2.792+/−0.25×40.011+/−0.45 ppm, 3.714+/−0.25 ppm×72.206+/−0.45 ppm, 3.009+/−0.25 ppm×32.551+/−0.45 ppm, 3.851+/−0.25 ppm×64.395+/−0.45 ppm, 7.534+/−0.25 ppm×134.842+/−0.45 ppm, 2.787+/−0.25 ppm×40.035+/−0.45 ppm, 6.914+/−0.25 ppm×115.449+/−0.45 ppm, 7.088+/−0.25 ppm×121.707+/−0.45 ppm, 3.714+/−0.25 ppm×72.229+/−0.45 ppm, 3.637+/−0.25 ppm×78.911+/−0.45 ppm, 6.974+/−0.25 ppm×120.651+/−0.45 ppm, 7.817+/−0.25 ppm×131.398+/−0.45 ppm, 7.534+/−0.25 ppm×129.772+/−0.45 ppm, 3.966+/−0.25 ppm×46.482+/−0.45 ppm, 3.01+/−0.25 ppm×32.525+/−0.45 ppm, 2.512+/−0.25 ppm×28.032+/−0.45 ppm, 7.533+/−0.25 ppm×131.372+/−0.45 ppm, 7.619+/−0.25 ppm×134.811+/−0.45 ppm, 3.722+/−0.25 ppm×75.654+/−0.45 ppm, 7.276+/−0.25 ppm×116.555+/−0.45 ppm, 5.016+/−0.25 ppm×74.051+/−0.45 ppm, 7.533+/−0.25 ppm×134.812+/−0.45 ppm, 7.086+/−0.25 ppm×121. 698+/−0.45 ppm, 7.817+/−0.25 ppm×131.389+/−0.45 ppm, 7.532+/−0.25 ppm×129.75+/−0.45 ppm, 3.969+/−0.25 ppm×46.484+/−0.45 ppm, 7.532+/−0.25 ppm×131.359+/−0.45 ppm, 7.618+/−0.25 ppm×134.804+/−0.45 ppm, 7.818+/−0.25 ppm×129.746+/−0.45 ppm, 9.117+/−0.25 ppm×148.321+/−0.45 ppm, 7.347+/−0.25 ppm×121. 578+/−0.45 ppm, 7.274+/−0.25 ppm×116.53+/−0.45 ppm, 2.711+/−0.25 ppm×47.702+/−0.45 ppm, in the ¹H and ¹³C dimensions respectively (Table 2).

Other known immune response biomarkers may be used in combination with the biomarkers of the present invention. In some embodiments, biomarker levels of the present invention are measured by determining the metabolite level of the biomarker in a biofluid. In certain aspects, metabolite levels of the biomarkers are determined using NMR spectroscopy or mass spectroscopy. In other embodiments, metabolite levels of the biomarkers are determined using immunoassay devices. One of the ordinary skills in the art has several methods and devices available for the detection and analysis of the markers of the invention.

Biomarkers of the present invention serve an important role in the early detection and monitoring of immune system response. Markers are typically substances found in a bodily sample that can be measured. The measured amount can correlate to underlying disease or disorder pathophysiology associated with altered immune system, presence or absence of disease or disorder due to altered immune system response, probability of a disease or disorder in the future due to altered immune system response. In patients receiving treatment for their condition the measured amount will also correlate with responsiveness to therapy. Accordingly, the methods of the present invention are useful for the differential diagnosis of diseases and disorders associated with the immune system.

### Clinical Assay Performance

The methods of the present invention may be used in clinical assays to diagnose or prognose an altered immune system response (ISR) in a subject, identify a subject at risk of a disease or disorder associated with altered ISR, and/or for prescribing a therapeutic regimen or predicting benefit from therapy in a subject having altered ISR. Clinical assay performance can be assessed by determining the assay's sensitivity, specificity and area under the ROC curve (AUC), accuracy, positive predictive value (PPV) and negative predictive value (NPV). Disclosed herein are assays for diagnosing or prognosing altered ISR in a subject, identifying a subject at risk of a disease or disorder associated with altered ISR, or for prescribing a therapeutic regimen or predicting benefit from therapy in a subject having altered ISR.

The clinical performance of the assay may be based on sensitivity. The sensitivity of an assay of the present invention may be at least about 40%, 45%, 50%, 55%, 60%, 65% 70%, 75%, 80%, 85%, 90%, 95%, 99% or 100%. The clinical performance of the assay may be based on specificity. The specificity of an assay of the present invention may be at least about 40%, 45%, 50%, 55%, 60%, 65% 70%, 75%, 80%, 85%, 90%, 95%, 99% or 100%. The clinical performance of the assay may be based on area under the ROC curve (AUC). The AUC of an assay of the present invention may be at least about 0.5, 0.55. 0.6, 0.65, 0.7, 0.75, 0.8, 0.85, 0.9, 0.95. The clinical performance of the assay may be based on accuracy. The accuracy of an assay of the present invention may be at least about 40%, 45%, 50%, 55%, 60%, 65% 70%, 75%, 80%, 85%, 90%, 95%, 99% or 100%.

### Compositions

Compositions useful in the methods of the present invention include compositions that specifically recognize a biomarker associated with altered ISR wherein the biomarker is choline, hippuric acid, indole-3-acetic acid, lysine, trigonelline, tryptophan, 2-pyrocatechuic-acid, 3-Hydroxymandelic acid,L-Phenylalanine,4-Methoxyphenylacetic acid, 4-Aminohippuric acid, Pteroyltriglutamic acid, 4-Ethylbenzoic acid, Aspartylphenylalanine, Creatinine, Diphenhydramine, D-Xylose, Gulonic acid, Hippuric acid, Homoveratric acid, Pyroglutamic acid, Quinic acid, Salicyluric acid, trigonelline, and metabolite resonances detected via 2D ¹H-¹³C HSQC NMR spectroscopy 1.275+/−0.25 ppm×30.8+/−0.45 ppm, 2.195+/−0.25 ppm×24.54+/−0.45 ppm, 2.22+/−0.25 ppm×39.75+/−0.45 ppm, 2.51+/−0.25 ppm×28.05+/−0.45 ppm, 2.825+/−0.25 ppm×30+/−0.45 ppm, 2.891+/−0.25 ppm×32.96+/−0.45 ppm, 3.119+/−0.25 ppm×32.76+/−0.45 ppm, 3.123+/−0.25 ppm×32.81+/−0.45 ppm, 3.166+/−0.25 ppm×44.04+/−0.45 ppm, 3.25+/−0.25 ppm×30.3+/−0.45 ppm, 3.38+/−0.25 ppm×76.2+/−0.45 ppm, 3.591+/−0.25 ppm×73.11+/−0.45 ppm, 3.62+/−0.25 ppm×78.2+/−0.45 ppm, 3.71+/−0.25 ppm×72.1+/−0.45 ppm, 3.717+/−0.25 ppm×69.98+/−0.45 ppm, 3.75+/−0.25 ppm×62+/−0.45 ppm, 3.9+/−0.25 ppm×79+/−0.45 ppm, 7.1+/−0.25 ppm×122+/−0.45 ppm, 7.533+/−0.25 ppm×129.7+/−0.45 ppm, 7.625+/−0.25 ppm×131.1+/−0.45 ppm, 7.65+/−0.25 ppm×119.85+/−0.45 ppm, 7.819+/−0.25 ppm×131.4+/−0.45 ppm, 3.876+/−0.25 ppm×35.932+/−0.45 ppm, 7.624+/−0.25 ppm×131.123+/−0.45 ppm, 7.532+/−0.25 ppm×134.804+/−0.45 ppm, 3.813+/−0.25 ppm×62.593+/−0.45 ppm, 1.991+/−0.25 ppm×40.132+/−0.45 ppm, 7.294+/−0.25 ppm×132.721+/−0.45 ppm, 4.44+/−0.25 ppm×50.942+/−0.45 ppm, 3.787+/−0.25 ppm×73.579+/−0.45 ppm, 6.914+/−0.25 ppm×115.425+/−0.45 ppm, 6.974+/−0.25 ppm×120.636+/−0.45 ppm, 7.085+/−0.25 ppm×121.7+/−0.45 ppm, 7.817+/−0.25 ppm×131. 402+/−0.45 ppm, 8.841+/−0.25 ppm×147.236+/−0.45 ppm, 3.969+/−0.25 ppm×46. 487+/−0.45 ppm, 8.08+/−0.25 ppm×130.252+/−0.45 ppm, 7.531+/−0.25 ppm×131. 363+/−0.45 ppm, 7.618+/−0.25 ppm×134.808+/−0.45 ppm, 7.818+/−0.25 ppm×129.762+/−0.45 ppm, 9.117+/−0.25 ppm×148. 323+/−0.45 ppm, 7.531+/−0.25 ppm×129.748+/−0.45 ppm, 7.346+/−0.25 ppm×121.584+/−0.45 ppm, 7.274+/−0.25 ppm×116.531+/−0.45 ppm, 2.566+/−0.25 ppm×47.724+/−0.45 ppm, 2.712+/−0.25 ppm×47.752+/−0.45 ppm, 2.792+/−0.25×40.011+/−0.45 ppm, 3.714+/−0.25 ppm×72.206+/−0.45 ppm, 3.009+/−0.25 ppm×32.551+/−0.45 ppm, 3.851+/−0.25 ppm×64.395+/−0.45 ppm, 7.534+/−0.25 ppm×134.842+/−0.45 ppm, 2.787+/−0.25 ppm×40.035+/−0.45 ppm, 6.914+/−0.25 ppm×115.449+/−0.45 ppm, 7.088+/−0.25 ppm×121.707+/−0.45 ppm, 3.714+/−0.25 ppm×72.229+/−0.45 ppm, 3.637+/−0.25 ppm×78.911+/−0.45 ppm, 6.974+/−0.25 ppm×120.651+/−0.45 ppm, 7.817+/−0.25 ppm×131.398+/−0.45 ppm, 7.534+/−0.25 ppm×129.772+/−0.45 ppm, 3.966+/−0.25 ppm×46.482+/−0.45 ppm, 3.01+/−0.25 ppm×32.525+/−0.45 ppm, 2.512+/−0.25 ppm×28.032+/−0.45 ppm, 7.533+/−0.25 ppm×131.372+/−0.45 ppm, 7.619+/−0.25 ppm×134.811+/−0.45 ppm, 3.722+/−0.25 ppm×75.654+/−0.45 ppm, 7.276+/−0.25 ppm×116.555+/−0.45 ppm, 5.016+/−0.25 ppm×74.051+/−0.45 ppm, 7.533+/−0.25 ppm×134.812+/−0.45 ppm, 7.086+/−0.25 ppm×121. 698+/−0.45 ppm, 7.817+/−0.25 ppm×131.389+/−0.45 ppm, 7.532+/−0.25 ppm×129.75+/−0.45 ppm, 3.969+/−0.25 ppm×46.484+/−0.45 ppm, 7.532+/−0.25 ppm×131.359+/−0.45 ppm, 7.618+/−0.25 ppm×134.804+/−0.45 ppm, 7.818+/−0.25 ppm×129.746+/−0.45 ppm, 9.117+/−0.25 ppm×148.321+/−0.45 ppm, 7.347+/−0.25 ppm×121. 578+/−0.45 ppm, 7.274+/−0.25 ppm×116.53+/−0.45 ppm, 2.711+/−0.25 ppm×47.702+/−0.45 ppm, in the ¹H and ¹³C dimensions respectively.

### Methods of Treatment

The present invention provides methods of treating diseases and disorder in a subject with altered ISR, comprising administering to the subject an effective amount of a composition, wherein the composition alters the levels of choline, hippuric acid, indole-3-acetic acid, lysine, trigonelline, tryptophan, 2-pyrocatechuic-acid, 3-Hydroxymandelic acid,L-Phenylalanine,4-Methoxyphenylacetic acid, 4-Aminohippuric acid, Pteroyltriglutamic acid, 4-Ethylbenzoic acid, Aspartylphenylalanine, Creatinine, Diphenhydramine, D-Xylose, Gulonic acid, Hippuric acid, Homoveratric acid, Pyroglutamic acid, Quinic acid, Salicyluric acid, trigonelline, and metabolite resonances at 1.275+/−0.25 ppm×30.8+/−0.45 ppm, 2.195+/−0.25 ppm×24.54+/−0.45 ppm, 2.22+/−0.25 ppm×39.75+/−0.45 ppm, 2.51+/−0.25 ppm×28.05+/−0.45 ppm, 2.825+/−0.25 ppm×30+/−0.45 ppm, 2.891+/−0.25 ppm×32.96+/−0.45 ppm, 3.119+/−0.25 ppm×32.76+/−0.45 ppm, 3.123+/−0.25 ppm×32.81+/−0.45 ppm, 3.166+/−0.25 ppm×44.04+/−0.45 ppm, 3.25+/−0.25 ppm×30.3+/−0.45 ppm, 3.38+/−0.25 ppm×76.2+/−0.45 ppm, 3.591+/−0.25 ppm×73.11+/−0.45 ppm, 3.62+/−0.25 ppm×78.2+/−0.45 ppm, 3.71+/−0.25 ppm×72.1+/−0.45 ppm, 3.717+/−0.25 ppm×69.98+/−0.45 ppm, 3.75+/−0.25 ppm×62+/−0.45 ppm, 3.9+/−0.25 ppm×79+/−0.45 ppm, 7.1+/−0.25 ppm×122+/−0.45 ppm, 7.533+/−0.25 ppm×129.7+/−0.45 ppm, 7.625+/−0.25 ppm×131.1+/−0.45 ppm, 7.65+/−0.25 ppm×119.85+/−0.45 ppm, 7.819+/−0.25 ppm×131.4+/−0.45 ppm, 3.876+/−0.25 ppm×35.932+/−0.45 ppm, 7.624+/−0.25 ppm×131.123+/−0.45 ppm, 7.532+/−0.25 ppm×134.804+/−0.45 ppm, 3.813+/−0.25 ppm×62.593+/−0.45 ppm, 1.991+/−0.25 ppm×40.132+/−0.45 ppm, 7.294+/−0.25 ppm×132.721+/−0.45 ppm, 4.44+/−0.25 ppm×50.942+/−0.45 ppm, 3.787+/−0.25 ppm×73.579+/−0.45 ppm, 6.914+/−0.25 ppm×115.425+/−0.45 ppm, 6.974+/−0.25 ppm×120.636+/−0.45 ppm, 7.085+/−0.25 ppm×121.7+/−0.45 ppm, 7.817+/−0.25 ppm×131. 402+/−0.45 ppm, 8.841+/−0.25 ppm×147.236+/−0.45 ppm, 3.969+/−0.25 ppm×46. 487+/−0.45 ppm, 8.08+/−0.25 ppm×130.252+/−0.45 ppm, 7.531+/−0.25 ppm×131. 363+/−0.45 ppm, 7.618+/−0.25 ppm×134.808+/−0.45 ppm, 7.818+/−0.25 ppm×129.762+/−0.45 ppm, 9.117+/−0.25 ppm×148. 323+/−0.45 ppm, 7.531+/−0.25 ppm×129.748+/−0.45 ppm, 7.346+/−0.25 ppm×121.584+/−0.45 ppm, 7.274+/−0.25 ppm×116.531+/−0.45 ppm, 2.566+/−0.25 ppm×47.724+/−0.45 ppm, 2.712+/−0.25 ppm×47.752+/−0.45 ppm, 2.792+/−0.25×40.011+/−0.45 ppm, 3.714+/−0.25 ppm×72.206+/−0.45 ppm, 3.009+/−0.25 ppm×32.551+/−0.45 ppm, 3.851+/−0.25 ppm×64.395+/−0.45 ppm, 7.534+/−0.25 ppm×134.842+/−0.45 ppm, 2.787+/−0.25 ppm×40.035+/−0.45 ppm, 6.914+/−0.25 ppm×115.449+/−0.45 ppm, 7.088+/−0.25 ppm×121.707+/−0.45 ppm, 3.714+/−0.25 ppm×72.229+/−0.45 ppm, 3.637+/−0.25 ppm×78.911+/−0.45 ppm, 6.974+/−0.25 ppm×120.651+/−0.45 ppm, 7.817+/−0.25 ppm×131.398+/−0.45 ppm, 7.534+/−0.25 ppm×129.772+/−0.45 ppm, 3.966+/−0.25 ppm×46.482+/−0.45 ppm, 3.01+/−0.25 ppm×32.525+/−0.45 ppm, 2.512+/−0.25 ppm×28.032+/−0.45 ppm, 7.533+/−0.25 ppm×131.372+/−0.45 ppm, 7.619+/−0.25 ppm×134.811+/−0.45 ppm, 3.722+/−0.25 ppm×75.654+/−0.45 ppm, 7.276+/−0.25 ppm×116.555+/−0.45 ppm, 5.016+/−0.25 ppm×74.051+/−0.45 ppm, 7.533+/−0.25 ppm×134.812+/−0.45 ppm, 7.086+/−0.25 ppm×121. 698+/−0.45 ppm, 7.817+/−0.25 ppm×131.389+/−0.45 ppm, 7.532+/−0.25 ppm×129.75+/−0.45 ppm, 3.969+/−0.25 ppm×46.484+/−0.45 ppm, 7.532+/−0.25 ppm×131.359+/−0.45 ppm, 7.618+/−0.25 ppm×134.804+/−0.45 ppm, 7.818+/−0.25 ppm×129.746+/−0.45 ppm, 9.117+/−0.25 ppm×148.321+/−0.45 ppm, 7.347+/−0.25 ppm×121. 578+/−0.45 ppm, 7.274+/−0.25 ppm×116.53+/−0.45 ppm, 2.711+/−0.25 ppm×47.702+/−0.45 ppm, in the ¹H and ¹³C dimensions respectively. In other embodiments, the present invention provides methods of treating a disease or disorder in a subject with altered ISR, comprising administering to the subject an effective amount of a composition that normalizes the level of choline, hippuric acid, indole-3-acetic acid, lysine, trigonelline, tryptophan, 2-pyrocatechuic-acid, 3-Hydroxymandelic acid,L-Phenylalanine,4-Methoxyphenylacetic acid, 4-Aminohippuric acid, Pteroyltriglutamic acid, 4-Ethylbenzoic acid, Aspartylphenylalanine, Creatinine, Diphenhydramine, D-Xylose, Gulonic acid, Hippuric acid, Homoveratric acid, Pyroglutamic acid, Quinic acid, Salicyluric acid, trigonelline, and metabolite resonances at 1.275+/−0.25 ppm×30.8+/−0.45 ppm, 2.195+/−0.25 ppm×24.54+/−0.45 ppm, 2.22+/−0.25 ppm×39.75+/−0.45 ppm, 2.51+/−0.25 ppm×28.05+/−0.45 ppm, 2.825+/−0.25 ppm×30+/−0.45 ppm, 2.891+/−0.25 ppm×32.96+/−0.45 ppm, 3.119+/−0.25 ppm×32.76+/−0.45 ppm, 3.123+/−0.25 ppm×32.81+/−0.45 ppm, 3.166+/−0.25 ppm×44.04+/−0.45 ppm, 3.25+/−0.25 ppm×30.3+/−0.45 ppm, 3.38+/−0.25 ppm×76.2+/−0.45 ppm, 3.591+/−0.25 ppm×73.11+/−0.45 ppm, 3.62+/−0.25 ppm×78.2+/−0.45 ppm, 3.71+/−0.25 ppm×72.1+/−0.45 ppm, 3.717+/−0.25 ppm×69.98+/−0.45 ppm, 3.75+/−0.25 ppm×62+/−0.45 ppm, 3.9+/−0.25 ppm×79+/−0.45 ppm, 7.1+/−0.25 ppm×122+/−0.45 ppm, 7.533+/−0.25 ppm×129.7+/−0.45 ppm, 7.625+/−0.25 ppm×131.1+/−0.45 ppm, 7.65+/−0.25 ppm×119.85+/−0.45 ppm, 7.819+/−0.25 ppm×131.4+/−0.45 ppm, 3.876+/−0.25 ppm×35.932+/−0.45 ppm, 7.624+/−0.25 ppm×131.123+/−0.45 ppm, 7.532+/−0.25 ppm×134.804+/−0.45 ppm, 3.813+/−0.25 ppm×62.593+/−0.45 ppm, 1.991+/−0.25 ppm×40.132+/−0.45 ppm, 7.294+/−0.25 ppm×132.721+/−0.45 ppm, 4.44+/−0.25 ppm×50.942+/−0.45 ppm, 3.787+/−0.25 ppm×73.579+/−0.45 ppm, 6.914+/−0.25 ppm×115.425+/−0.45 ppm, 6.974+/−0.25 ppm×120.636+/−0.45 ppm, 7.085+/−0.25 ppm×121.7+/−0.45 ppm, 7.817+/−0.25 ppm×131. 402+/−0.45 ppm, 8.841+/−0.25 ppm×147.236+/−0.45 ppm, 3.969+/−0.25 ppm×46. 487+/−0.45 ppm, 8.08+/−0.25 ppm×130.252+/−0.45 ppm, 7.531+/−0.25 ppm×131. 363+/−0.45 ppm, 7.618+/−0.25 ppm×134.808+/−0.45 ppm, 7.818+/−0.25 ppm×129.762+/−0.45 ppm, 9.117+/−0.25 ppm×148. 323+/−0.45 ppm, 7.531+/−0.25 ppm×129.748+/−0.45 ppm, 7.346+/−0.25 ppm×121.584+/−0.45 ppm, 7.274+/−0.25 ppm×116.531+/−0.45 ppm, 2.566+/−0.25 ppm×47.724+/−0.45 ppm, 2.712+/−0.25 ppm×47.752+/−0.45 ppm, 2.792+/−0.25×40.011+/−0.45 ppm, 3.714+/−0.25 ppm×72.206+/−0.45 ppm, 3.009+/−0.25 ppm×32.551+/−0.45 ppm, 3.851+/−0.25 ppm×64.395+/−0.45 ppm, 7.534+/−0.25 ppm×134.842+/−0.45 ppm, 2.787+/−0.25 ppm×40.035+/−0.45 ppm, 6.914+/−0.25 ppm×115.449+/−0.45 ppm, 7.088+/−0.25 ppm×121.707+/−0.45 ppm, 3.714+/−0.25 ppm×72.229+/−0.45 ppm, 3.637+/−0.25 ppm×78.911+/−0.45 ppm, 6.974+/−0.25 ppm×120.651+/−0.45 ppm, 7.817+/−0.25 ppm×131.398+/−0.45 ppm, 7.534+/−0.25 ppm×129.772+/−0.45 ppm, 3.966+/−0.25 ppm×46.482+/−0.45 ppm, 3.01+/−0.25 ppm×32.525+/−0.45 ppm, 2.512+/−0.25 ppm×28.032+/−0.45 ppm, 7.533+/−0.25 ppm×131.372+/−0.45 ppm, 7.619+/−0.25 ppm×134.811+/−0.45 ppm, 3.722+/−0.25 ppm×75.654+/−0.45 ppm, 7.276+/−0.25 ppm×116.555+/−0.45 ppm, 5.016+/−0.25 ppm×74.051+/−0.45 ppm, 7.533+/−0.25 ppm×134.812+/−0.45 ppm, 7.086+/−0.25 ppm×121. 698+/−0.45 ppm, 7.817+/−0.25 ppm×131.389+/−0.45 ppm, 7.532+/−0.25 ppm×129.75+/−0.45 ppm, 3.969+/−0.25 ppm×46.484+/−0.45 ppm, 7.532+/−0.25 ppm×131.359+/−0.45 ppm, 7.618+/−0.25 ppm×134.804+/−0.45 ppm, 7.818+/−0.25 ppm×129.746+/−0.45 ppm, 9.117+/−0.25 ppm×148.321+/−0.45 ppm, 7.347+/−0.25 ppm×121. 578+/−0.45 ppm, 7.274+/−0.25 ppm×116.53+/−0.45 ppm, 2.711+/−0.25 ppm×47.702+/−0.45 ppm, in the ¹H and ¹³C dimensions respectively.

### Kits

Another aspect of the invention encompasses kits for detecting or monitoring a altered immune response in a subject. A variety of kits having different components are contemplated by the current invention. Generally speaking, the kit will include the means for quantifying one or more biomarkers in a subject. In another embodiment, the kit will include means for collecting a biological sample, means for quantifying one or more biomarkers in the biological sample, and instructions for use of the kit contents. In certain embodiments, the kit comprises a means for quantifying the amount of a biomarker. In further aspects, the means for quantifying the amount of a biomarker comprises reagents necessary to detect the amount of a biomarker.

In one embodiment of the kit, means for collecting urine samples from patients that have been diagnosed with a disease or disorder associated with altered ISR or increased risk of a disease or disorder associated with altered ISR, which disease or disorder is selected from the group consisting of: infectious and inflammatory disorders, allergic and autoimmune diseases will be included. Means for quantifying the urine samples will be done by NMR spectroscopy, two-dimension NMR spectroscopy, or mass spectrometry, or some combination of one-dimensional NMR spectroscopy, two-dimensional NMR spectroscopy, and mass spectrometry. In some embodiments, the method for quantification will be heteronuclear single-quantum correlation (HSQC) two-dimensional NMR spectroscopy. In some embodiments, the method for quantification will be ¹H-¹³C heteronuclear single-quantum correlation (HSQC) two-dimensional NMR spectroscopy.

TABLE 1

| Metabolite | Description |
|---|---|
| Choline | Choline is an essential nutrient |
| | obtained from choline |
| | phospholipids such as |
| | phosphatidylcholine. It is classified |
| | as a nutrient with an amino acid- |
| | like metabolism and choline |
| | phospholipids are necessary |
| | components of cell membranes. |
| | Certain molecules like the |
| | neurotransmitter acetylcholine and |
| | the homocysteine precursor, |
| | adenosylmethionine, require choline |
| | for production (Zeisel, 2000). |
| Hippuric acid | Hippuric acid (HA) is a normal |
| | urinary component derived from the |
| | degradation of plant (poly)phenols |
| | and aromatic amino acids |
| | (phenylalanine and tryptophan) by |
| | the intestinal microbiota (Pero, |
| | 2010). It is produced by the |
| | conjugation of benzoic acid with |
| | glycine, a reaction that occurs in |
| | liver and kidneys (Wikoff et al. |
| | 2008). |
| Indole-3-acetic acid | Indole-3-acetic acid (IAA) is a |
| | naturally occurring plant hormone |
| | and can be synthesized from |
| | tryptophan by gut microbiota. Its |
| | mechanism of action is not yet fully |
| | understood. |
| Lysine | Lysine is an essential proteogenic |
| | amino acid, important for protein |
| | synthesis, crosslinking of collagen |
| | peptides, uptake of nutrients, and |
| | the production of carnitine. Lysine |
| | is synthesized by plants and bacteria |
| | via the diaminopimelate and a- |
| | aminoadipate pathways, and most |
| | catabolized through the saccharine |
| | pathway (Hall, 2018). |
| Trigonelline | Trigonelline (TRG) also known as |
| | beatine nicotinate, is found in |
| | fenugreek leaves and coffee |
| | (Chowdhury, 2018). TRG can be |
| | synthesized by intestinal microbiota |
| | during the conversion of S- |
| | adenozylmethionine to S- |
| | adenosylhomocysteine and is a |
| | product of the metabolism of niacin |
| | (vitamin B3). |
| Tryptophan | Tryptophan (Trp) is an essential |
| | proteogenic amino acid. It is the |
| | precursor to the neurotransmitter |
| | and platelet clotting factor |
| | serotonin, and the hormone |
| | melatonin (Slominski, 2002). It is |
| | also a precursor vitamin B3 which |
| | is synthesized via kynureine and |
| | quinolinic acids and aids in the |
| | conversion of carbohydrates to |
| | glucose (Buczko, 2005). It is also |
| | the sole source for production of |
| | NAD+ and NADP+. |
| 2-Pyrocatechuic Acid | 2-Pyrocatechuic Acid is a normal |
| | human benzoic acid metabolite |
| | (Jiye Jiye, 2005). It has been |
| | suggested that 2-Pyrocatechuic acid |
| | can serve as a biomarker for the |
| | detection and quantification of OH |
| | radicals. The role of OH radicals |
| | has been studied for involvement in |
| | the production of local and systemic |
| | tissue diseases, specifically those |
| | that manifest as pain disorders |
| | (Haque, 1994). |
| 3-Hydroxymandelic acid | 3-Hydroxymandelic acid is derived |
| | from mandelic acid through the |
| | mandelate pathway. The acid has |
| | been mapped to kidney disease as a |
| | potential biomarker (National |
| | Center for Biotechnology |
| | Information PubChem, 2022. |
| L-Phenylalanine | L-Phenylalanine is the L- |
| | enantiomer of phenylalanine and is |
| | an essential amino acid (National |
| | Center for Biotechnology |
| | Information PubChem, 2022). |
| | Phenylalanine is critical for the |
| | biosynthesis of other amino acids |
| | and the structure and function of |
| | proteins and enzymes. L- |
| | phenylalanine is a nutraceutical and |
| | a micronutrient. Phenylalanine is |
| | converted to tyrosine, and |
| | impairment of phenylalanine |
| | conversion to tyrosine is seen in |
| | chronic kidney disease (Kopple, |
| | 2007; Druml, 1989.). |
| 4-Methoxyphenylacetic acid | 4-Methoxyphenylacetic acid is |
| | phenylacetic acid with a 4-methoxy |
| | substituent. It is monocarboxylic. |
| | Anticancer and |
| | antineuroinflammatory effects have |
| | been discovered via bioassays |
| | (National Center for Biotechnology |
| | Information PubChem, 2022). |
| 4-Aminohippuric acid | Aminohippurate is the glycine |
| | amide of p-aminobenzoic acid. |
| | Aminohippuric acid is filtered by |
| | the glomeruli in the kidney and |
| | secreted by the proximal tubules. It |
| | is used as a diagnostic agent in the |
| | measurement of renal plasma |
| | flow.(National Center for |
| | Biotechnology Information |
| | PubChem, 2022) |
| Pteroyltriglutamic acid | Pteroyltriglutamic acid is a |
| | crystalline conjugate of folic acid |
| | with three molecules of glutamic |
| | acid. It has the properties of a |
| | polypeptide (HMDB, 2022)). |
| | Several studies on acute tubular |
| | necrosis have pointed towards a role |
| | of pteroyltrigultamic acid in |
| | intratubular deposit formations in |
| | the kidney and fibroblastic lesions |
| | (Mullin, 1976; Lee. 1952. |
| 4-Ethylbenzoic acid | 4-Ethylbenzoic acid, also known as 4- |
| | ethylbenzoate, belongs to the class of |
| | organic compounds known as benzoic |
| | acids. It is used in chemical synthesis |
| | of polymers and oilier molecules. |
| Aspartylphenylalanine | Aspartylphenylalnine is a dipeptide |
| | comprised of aspartate and |
| | phenylalanine and is a metabolic |
| | byproduct of aspartame. Several |
| | Asp-Phe dipeptidases degrade this |
| | peptide, and it has been suggested |
| | individuals with aspartame allergies |
| | may be deficient in degrading |
| | peptidases. A breakdown product of |
| | aspartylphenyalanine, N-beta- |
| | aspartylphenyalanine is a naturally |
| | occurring peptide found in blood |
| | and urine. |
| Creatinine | Creatine, excreted via urine as |
| | creatinine, is an endogenous amino |
| | acid that occurs primarily in muscle |
| | cells. It plays a main role in energy |
| | storage and conversion of ADP to |
| | ATP. It has been associated in the |
| | literature with lactic acidosis, acute |
| | kidney injury, atrial fibrillation, and |
| | arthritis, among other diseases and |
| | disorders (National Center for |
| | Biotechnology Information |
| | PubChem, 2022). |
| Diphenhydramine | Diphenhydramine is a |
| | pharmaceutical used as an |
| | antihistamine, antitussive, and |
| | sedative-hypnotic drugs. |
| D-Xylose | Xylose is found in |
| | mucopolysaccharides and the urine. |
| | It is one of the eight sugars that are |
| | essential for human nutrition. It is |
| | the primary sugar addition to serine |
| | or threonine residues during |
| | proteoglycan type O-glycosylation |
| | (HMDB, 2022) |
| Gulonic Acid | Gulonic acid is a primary |
| | metabolite. Gulonate, the conjugate |
| | base of L-gulonic acid, has been |
| | identified as a metabolite biomarker |
| | for the progression of chronic |
| | kidney disease in children |
| | (Denburg, 2021). |
| Homoveratric Acid | Homoveratric acid is a human |
| | urinary metabolite and human |
| | xenobiotic metabolite. It is a |
| | member of the phenylacetic acids. |
| Pyroglutamic Acid | Pyroglutamic acid is a natural |
| | amino acid derivative that has a free |
| | amino acid group of glutamic acid |
| | or glutamine cyclized that form a |
| | lactam |
| | (https://www.jstor.org/stable/24083854). |
| | It may function in glutamate |
| | storage. It acts on the cholinergic |
| | system in the brain and opposes the |
| | action of glutamate in the brain and |
| | body. Pyroglutamic acid within |
| | Amyloid β has been implicated in |
| | the progression of Alzheimer's |
| | Disease (Jawhar, 2011). Increased |
| | levels of this acid in the blood can |
| | occur due to inborn errors of |
| | metabolism. |
| Quinic Acid | Quinic acid is a crystalline sugar |
| | acid. It has been detected in a |
| | number of cancers, including |
| | prostate, colorectal, and bladder |
| | cancer in addition to colorectal |
| | adenomas (HMDB, 2022)). |
| Saliclyuric Acid | Saliclyuric acid is the gylcine |
| | conjugate of salicylic acid. |
| | Salicyluric acid is excreted from the |
| | body via the kidneys and is a |
| | uremic toxin. (National National |
| | Institute for Biotechnology |
| | Information PubMeb, 2022) |

```
TABLE 2
Metabolite Resonances
1.275 +/− 0.25 ppm × 30.8 +/− 0.45 ppm
2.195 +/− 0.25 ppm × 24.54 +/− 0.45 ppm
2.22 +/− 0.25 ppm × 39.75 +/− 0.45 ppm
2.51 +/− 0.25 ppm × 28.05 +/− 0.45 ppm
2.825 +/− 0.25 ppm × 30 +/− 0.45 ppm
2.891 +/− 0.25 ppm × 32.96 +/− 0.45 ppm
3.119 +/− 0.25 ppm × 32.76 +/− 0.45 ppm
3.123 +/− 0.25 ppm × 32.81 +/− 0.45 ppm
3.166 +/− 0.25 ppm × 44.04 +/− 0.45 ppm
3.25 +/− 0.25 ppm × 30.3 +/− 0.45 ppm
3.38 +/− 0.25 ppm × 76.2 +/− 0.45 ppm
3.591 +/− 0.25 ppm × 73.11 +/− 0.45 ppm
3.62 +/− 0.25 ppm × 78.2 +/− 0.45 ppm
3.71 +/− 0.25 ppm × 72.1 +/− 0.45 ppm
3.717 +/− 0.25 ppm × 69.98 +/− 0.45 ppm
3.75 +/− 0.25 ppm × 62 +/− 0.45 ppm
3.9 +/− 0.25 ppm × 79 +/− 0.45 ppm
7.1 +/− 0.25 ppm × 122 +/− 0.45 ppm
7.533 +/− 0.25 ppm × 129.7 +/− 0.45 ppm
7.625 +/− 0.25 ppm × 131.1 +/− 0.45 ppm
7.65 +/− 0.25 ppm × 119.85 +/− 0.45 ppm
7.819 +/− 0.25 ppm × 131.4 +/− 0.45 ppm
3.876 +/− 0.25 ppm × 35.932 +/− 0.45 ppm
7.624 +/− 0.25 ppm × 131.123 +/− 0.45 ppm
7.532 +/− 0.25 ppm × 134.804 +/− 0.45 ppm
3.813 +/− 0.25 ppm × 62.593 +/− 0.45 ppm
1.991 +/− 0.25 ppm × 40.132 +/− 0.45 ppm
7.294 +/− 0.25 ppm × 132.721 +/− 0.45 ppm
4.44 +/− 0.25 ppm × 50.942 +/− 0.45 ppm
3.787 +/− 0.25 ppm × 73.579 +/− 0.45 ppm
6.914 +/− 0.25 ppm × 115.425 +/− 0.45 ppm
6.974 +/− 0.25 ppm × 120.636 +/− 0.45 ppm
7.085 +/− 0.25 ppm × 121.7 +/− 0.45 ppm
7.817 +/− 0.25 ppm × 131.402 +/− 0.45 ppm
8.841 +/− 0.25 ppm × 147.236 +/− 0.45 ppm
3.969 +/− 0.25 ppm × 46.487 +/− 0.45 ppm
  8.08 +/− 0.25 ppm × 130.252 +/− 0.45 ppm
7.531 +/− 0.25 ppm × 131.363 +/− 0.45 ppm
7.618 +/− 0.25 ppm × 134.808 +/− 0.45 ppm
7.818 +/− 0.25 ppm × 129.762 +/− 0.45 ppm
9.117 +/− 0.25 ppm × 148.323 +/− 0.45 ppm
7.531 +/− 0.25 ppm × 129.748 +/− 0.45 ppm
7.346 +/− 0.25 ppm × 121.584 +/− 0.45 ppm
7.274 +/− 0.25 ppm × 116.531 +/− 0.45 ppm
2.566 +/− 0.25 ppm × 47.724 +/− 0.45 ppm
2.712 +/− 0.25 ppm × 47.752 +/− 0.45 ppm
  2.792 +/− 0.25 × 40.011 +/− 0.45 ppm
3.714 +/− 0.25 ppm × 72.206 +/− 0.45 ppm
3.009 +/− 0.25 ppm × 32.551 +/− 0.45 ppm
3.851 +/− 0.25 ppm × 64.395 +/− 0.45 ppm
7.534 +/− 0.25 ppm × 134.842 +/− 0.45 ppm
2.787 +/− 0.25 ppm × 40.035 +/− 0.45 ppm
6.914 +/− 0.25 ppm × 115.449 +/− 0.45 ppm
7.088 +/− 0.25 ppm × 121.707 +/− 0.45 ppm
3.714 +/− 0.25 ppm × 72.229 +/− 0.45 ppm
3.637 +/− 0.25 ppm × 78.911 +/− 0.45 ppm
6.974 +/− 0.25 ppm × 120.651 +/− 0.45 ppm
7.817 +/− 0.25 ppm × 131.398 +/− 0.45 ppm
7.534 +/− 0.25 ppm × 129.772 +/− 0.45 ppm
3.966 +/− 0.25 ppm × 46.482 +/− 0.45 ppm
3.01 +/− 0.25 ppm × 32.525 +/− 0.45 ppm
2.512 +/− 0.25 ppm × 28.032 +/− 0.45 ppm
7.533 +/− 0.25 ppm × 131.372 +/− 0.45 ppm
7.619 +/− 0.25 ppm × 134.811 +/− 0.45 ppm
3.722 +/− 0.25 ppm × 75.654 +/− 0.45 ppm
7.276 +/− 0.25 ppm × 116.555 +/− 0.45 ppm
5.016 +/− 0.25 ppm × 74.051 +/− 0.45 ppm
7.533 +/− 0.25 ppm × 134.812 +/− 0.45 ppm
7.086 +/− 0.25 ppm × 121.698 +/− 0.45 ppm
7.817 +/− 0.25 ppm × 131.389 +/− 0.45 ppm
7.532 +/− 0.25 ppm × 129.75 +/− 0.45 ppm
3.969 +/− 0.25 ppm × 46.484 +/− 0.45 ppm
7.532 +/− 0.25 ppm × 131.359 +/− 0.45 ppm
7.618 +/− 0.25 ppm × 134.804 +/− 0.45 ppm
7.818 +/− 0.25 ppm × 129.746 +/− 0.45 ppm
9.117 +/− 0.25 ppm × 148.321 +/− 0.45 ppm
7.347 +/− 0.25 ppm × 121.578 +/− 0.45 ppm
7.274 +/− 0.25 ppm × 116.53 +/− 0.45 ppm
2.711 +/− 0.25 ppm × 47.702 +/− 0.45 ppm
```

### EXAMPLES

The following examples are put forth to provide those of ordinary skill in the art with a complete disclosure and description of how to make and use the present invention and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

The invention will be further understood by reference to the following examples, which are intended to be purely exemplary of the invention. These examples are provided solely to illustrate in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description. Such modifications are intended to fall within the scope of the appended claims.

### Example 1-71

Metabolite Urine Levels in Human Kidney Transplant Subjects with Altered Immune System Response (ISR) Who Develop BK Virus Interstitial Nephritis (BKVIN).

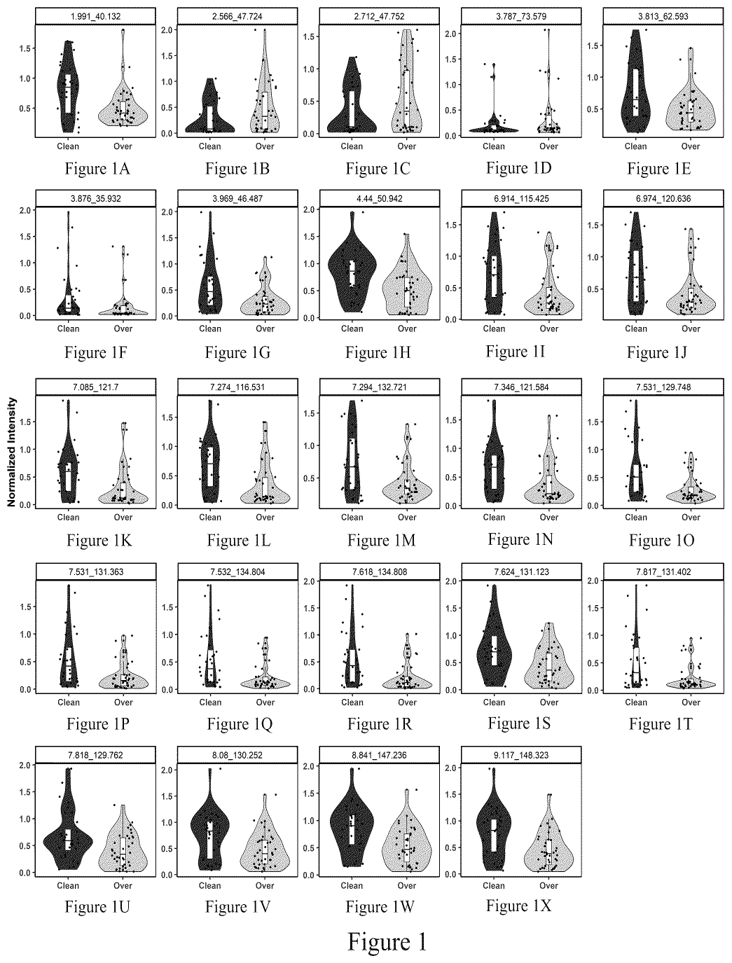

Levels of metabolites were assayed in human kidney transplant subjects who developed BKVIN and kidney transplant patients who had a stable graft (controls) as follows. Urine was collected from control subjects (N=32) and subjects who would develop BKVIN (N=39). Urine was centrifuged stored in aliquots at −80° C. Aliquots were processed for metabolite extraction and metabolite levels were measured by NMR spectroscopy. Using NMR spectroscopy levels of metabolite resonances at 3.876+/−0.25 ppm×35.932+/−0.45 ppm, 7.624+/−0.25 ppm×131.123+/−0.45 ppm, 7.532+/−0.25 ppm×134.804+/−0.45 ppm, 3.813+/−0.25 ppm×62.593+/−0.45 ppm, 1.991+/−0.25 ppm×40.132+/−0.45 ppm, 7.294+/−0.25 ppm×132.721+/−0.45 ppm, 4.44+/−0.25 ppm×50.942+/−0.45 ppm, 3.787+/−0.25 ppm×73.579+/−0.45 ppm, 6.914+/−0.25 ppm×115.425+/−0.45 ppm, 6.974+/−0.25 ppm×120.636+/−0.45 ppm, 7.085+/−0.25 ppm×121.7+/−0.45 ppm, 7.817+/−0.25 ppm×131.402+/−0.45 ppm, 8.841+/−0.25 ppm×147.236+/−0.45 ppm, 3.969+/−0.25 ppm×46.487+/−0.45 ppm, 8.08+/−0.25 ppm×130.252+/−0.45 ppm, 7.531+/−0.25 ppm×131.363+/−0.45 ppm, 7.618+/−0.25 ppm×134.808+/−0.45 ppm, 7.818+/−0.25 ppm×129.762+/−0.45 ppm, 9.117+/−0.25 ppm×148.323+/−0.45 ppm, 7.531+/−0.25 ppm×129.748+/−0.45 ppm, 7.346+/−0.25 ppm×121.584+/−0.45 ppm, 7.274+/−0.25 ppm×116.531+/−0.45 ppm, 2.566+/−0.25 ppm×47.724+/−0.45 ppm, 2.712+/−0.25 ppm×47.752+/−0.45 ppm in the ¹H and ¹³C dimensions respectively were significantly different between BKVIN and control subjects (See FIG. 1 consisting of panels 1A-1X).

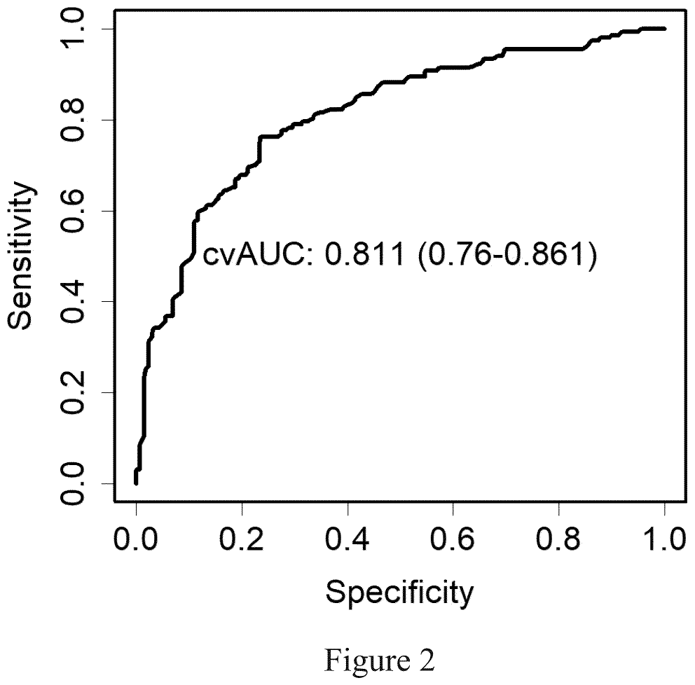

Using the differential levels of the above-mentioned metabolites, a machine learning algorithm produced a biomarker of response (BoR) score that differentiates kidney transplant subjects that develop BKVIN from controls with 81.1% cross-validated AUC (cvAUC) (see FIG. 2).

These results showed that metabolite biomarkers in urine are useful for identifying kidney transplant patients at risk for developing BKVIN. These results further indicated that methods and biomarkers of the present invention are useful for diagnosing BKVIN, patients who are at risk for BKVIN and other diseases or disorders associated with altered immune system response.

### Example 72-121

Metabolite Urine Levels in Human Kidney Transplant Subjects with Altered Immune System Response (ISR) Who Develop Graft Dysfunction, Rejection, or Failure.

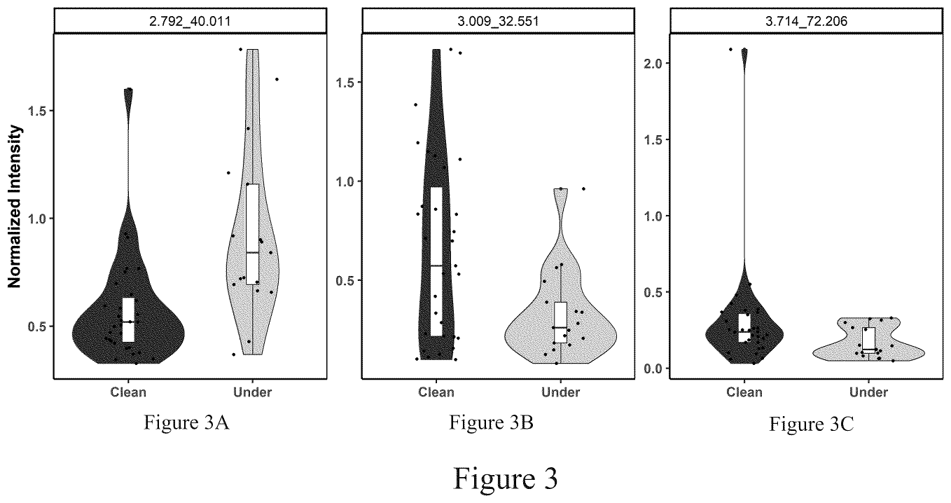

Levels of metabolites were assayed in human kidney transplant subjects who developed donor specific antibodies, anti-body-mediated rejection (AMR). T-cell mediated rejection or other forms of graft dysfunction or rejection associated with under-immunosuppression (“under-immunosuppressed” N=17) and kidney transplant patients who had a stable graft (“controls”, N=32) as follows. Urine was collected from all subjects, centrifuged stored in aliquots at −80° C. Aliquots were processed for metabolite extraction and metabolite levels were measured by NMR spectroscopy. Using NMR spectroscopy levels metabolite resonances at 2.792+/−0.25 ppm×40.011+/−0.45 ppm, 3.714+/−0.25 ppm×72.206+/−0.45 ppm, 3.009+/−0.25 ppm×32.551+/−0.45 ppm in the ¹H and ¹³C dimensions respectively were significantly different between subjects who were under-immunosuppressed and control subjects (See FIG. 3 consisting of panels 3A-3C).

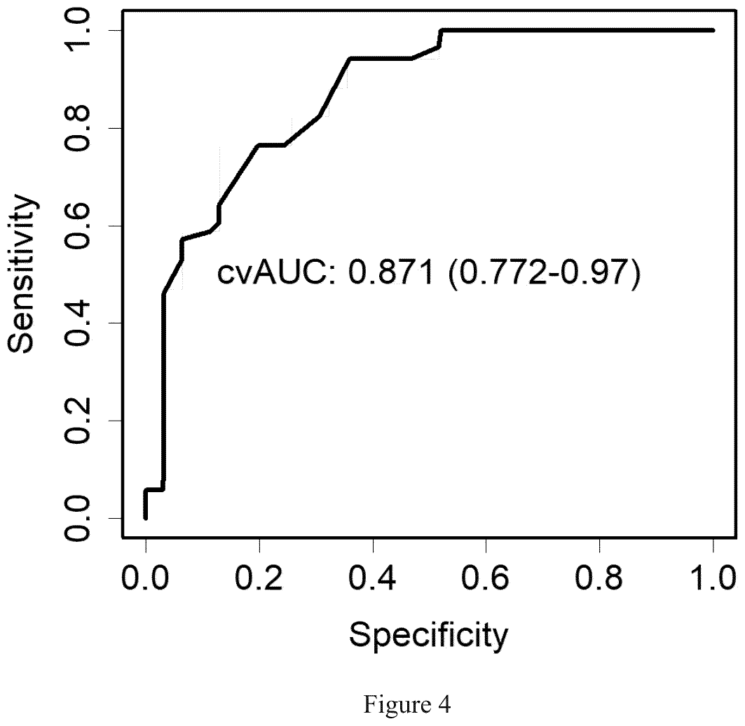

Using the differential levels of the above-mentioned metabolites, a machine learning algorithm produced a biomarker of response (BoR) score that differentiates kidney transplant subjects that were under-immunosuppressed from controls with 87.1% cross-validated AUC (cvAUC) (see FIG. 4).

These results showed that metabolite biomarkers in urine are useful for identifying kidney transplant patients at risk for under-immunosuppression events like rejection, graft dysfunction and graft loss, These results further indicated that methods and biomarkers of the present invention are useful for diagnosing graft dysfunction, rejection or failure and other diseases or disorders associated with altered immune system response.

### Example 122-176

Metabolite Urine Levels in Human Kidney Transplant Subjects with Altered Immune System Response (ISR) Who Develop Graft Dysfunction, Rejection, or Failure due to under-immunosuppression and Subjects with Altered Immune System Response (ISR) Who Develop BK Virus Interstitial Nephritis (BKVIN) due to over-immunosuppression.

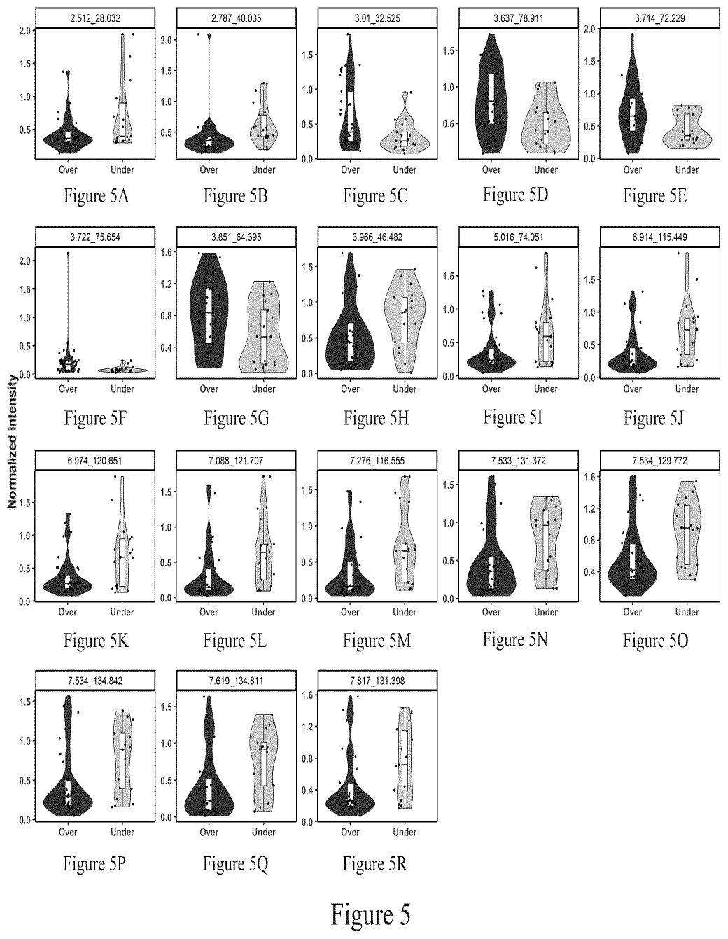

Levels of metabolites were assayed in human kidney transplant subjects who developed donor specific antibodies, anti-body-mediated rejection (AMR). T-cell mediated rejection or other forms of graft dysfunction or rejection associated with under-immunosuppression (“under-immunosuppressed” N=17) and subjects who would develop BKVIN (“over-immunosuppressed” N=37). Urine was collected from all subjects, centrifuged stored in aliquots at −80° C. Aliquots were processed for metabolite extraction and metabolite levels were measured by NMR spectroscopy. Using NMR spectroscopy levels of metabolite resonances at 3.851+/−0.25 ppm×64.395+/−0.45 ppm, 7.534+/−0.25 ppm×134.842+/−0.45 ppm, 2.787+/−0.25 ppm×40.035+/−0.45 ppm, 6.914+/−0.25 ppm×115.449+/−0.45 ppm, 7.088+/−0.25 ppm×121.707+/−0.45 ppm, 3.714+/−0.25 ppm×72.229+/−0.45 ppm, 3.637+/−0.25 ppm×78.911+/−0.45 ppm, 6.974+/−0.25 ppm×120.651+/−0.45 ppm, 7.817+/−0.25 ppm×131.398+/−0.45 ppm, 7.534+/−0.25 ppm×129.772+/−0.45 ppm, 3.966+/−0.25 ppm×46.482+/−0.45 ppm, 3.01+/−0.25 ppm×32.525+/−0.45 ppm, 2.512+/−0.25 ppm×28.032+/−0.45 ppm, 7.533+/−0.25 ppm×131.372+/−0.45 ppm, 7.619+/−0.25 ppm×134.811+/−0.45 ppm, 3.722+/−0.25 ppm×75.654+/−0.45 ppm, 7.276+/−0.25 ppm×116.555+/−0.45 ppm, 5.016+/−0.25 ppm×74.051+/−0.45 ppm in the ¹H and ¹³C dimensions respectively were significantly different between under and over-immunosuppressed subjects (See FIG. 5 consisting of panels 5A-5R).

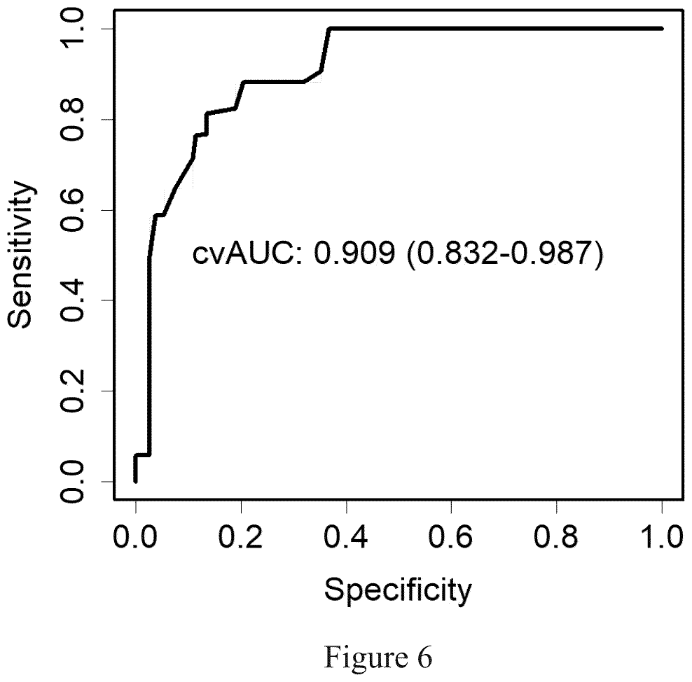

Using the differential levels of the above-mentioned metabolites, a machine learning algorithm produced a biomarker of response (BoR) score that differentiates under and over-immunosuppressed subjects with 90.9% cross-validated AUC (cvAUC) (see FIG. 6).

These results showed unique metabolite biomarkers in urine which are useful for identifying kidney transplant patients at risk for both under and over-immunosuppression complications. These results further indicated that methods and biomarkers of the present invention are useful for diagnosing graft dysfunction, rejection or failure and other diseases or disorders associated with altered immune system response.

### Example 177-262

Metabolite Urine Levels in Human Kidney Transplant Subjects with Appropriate Immune System Response (ISR).

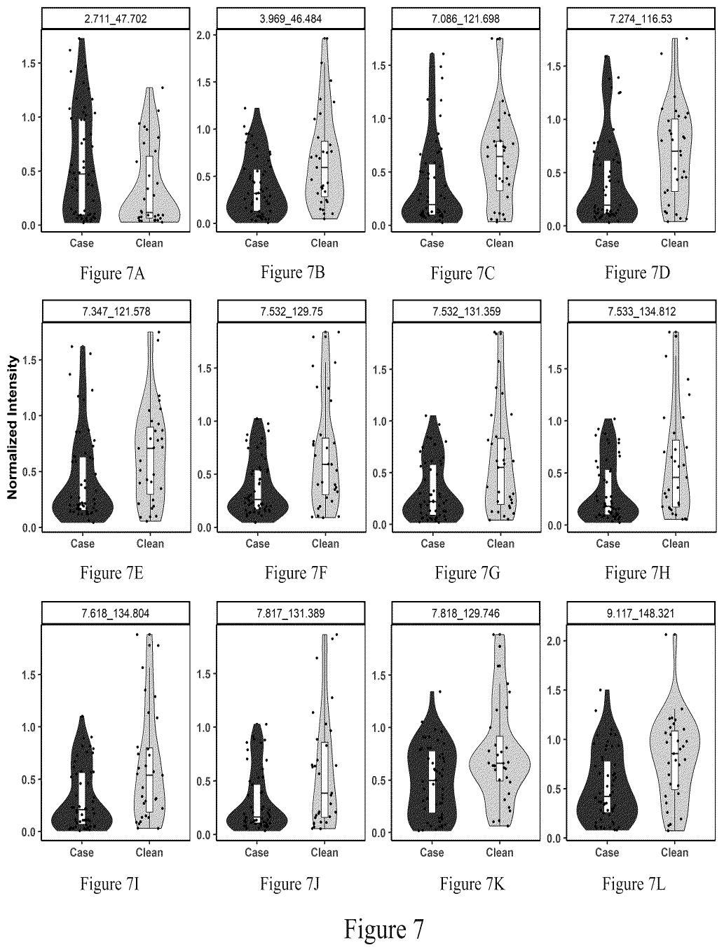

Levels of metabolites were assayed in human kidney transplant subjects who had a stable graft for 2 years with no signs of ISR (either rejection or infection) (N=31 controls) and compared to patients who had ISR demonstrated by either under-immunosuppression (ie donor specific antibodies or a rejection event) or over-immunosuppression (ie infection or BKVIN) (N=54). Urine was collected from all subjects. centrifuged stored in aliquots at −80° C. Aliquots were processed for metabolite extraction and metabolite levels were measured by NMR spectroscopy. Using NMR spectroscopy levels of metabolite resonances at 2.711+/−0.25 ppm×47.702+/−0.45 ppm, 3.969+/−0.25 ppm×46.484+/−0.45 ppm, 7.086+/−0.25 ppm×121.698+/−0.45 ppm, 7.274+/−0.25 ppm×116.53+/−0.45 ppm, 7.347+/−0.25 ppm×121.578+/−0.45 ppm, 7.532+/−0.25 ppm×129.75+/−0.45 ppm, 7.532+/−0.25 ppm×131.359+/−0.45 ppm, 7.533+/−0.25 ppm×134.812+/−0.45 ppm, 7.618+/−0.25 ppm×134.804+/−0.45 ppm, 7.817+/−0.25 ppm×131.389+/−0.45 ppm, 7.818+/−0.25 ppm×129.746+/−0.45 ppm, 9.117+/−0.25 ppm×148.321+/−0.45 ppm in the ¹H and ¹³C dimensions respectively were significantly different between subjects who had appropriate ISR compared to those that did not (See FIG. 7 consisting of panels 7A-7L).

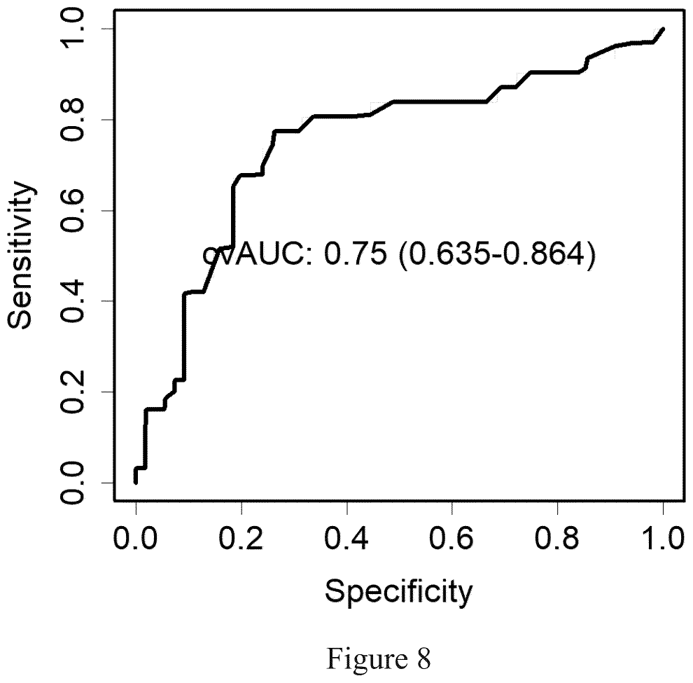

Using the differential levels of the above-mentioned metabolites, a machine learning algorithm produced a biomarker of response (BoR) score that differentiates kidney transplant with appropriate ISR with 75% cross-validated AUC (cvAUC) (see FIG. 8).

These results showed that metabolite biomarkers in urine are useful for identifying kidney transplant patients who have a well-functioning immune systems and are not at risk for complications due to under or over-immunosuppression. These results further indicated that methods and biomarkers of the present invention are useful for diagnosing diseases or disorders associated with altered immune system response.

### Example 263-302

Metabolite Urine Levels in Human Kidney Transplant Subjects with Altered Immune System Response (ISR) Who Develop BK Virus Interstitial Nephritis (BKVIN).

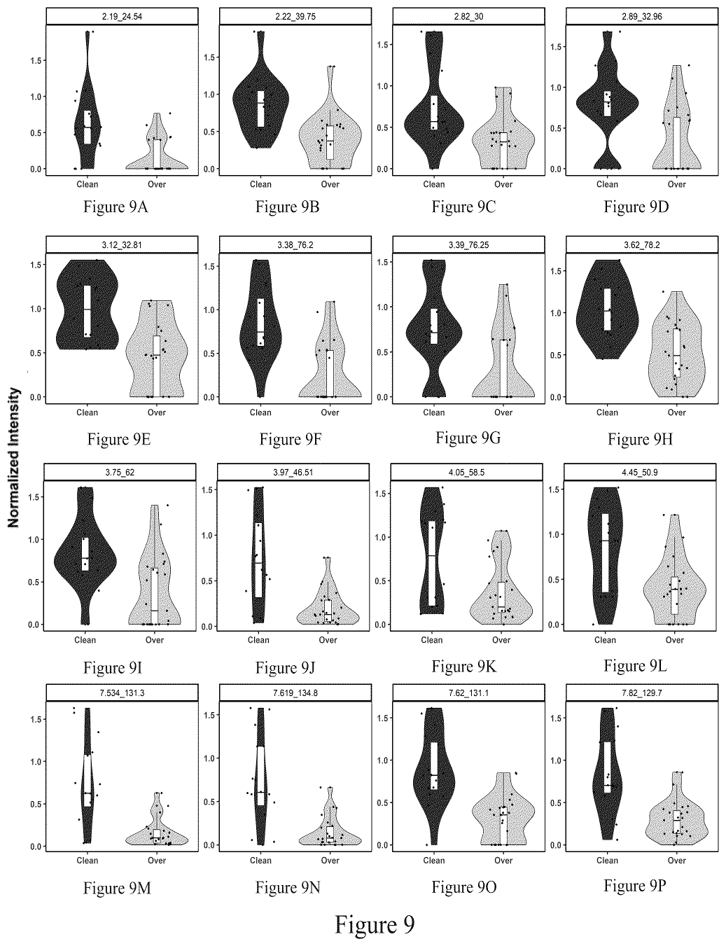

In an additional cohort levels of metabolites were assayed as described in examples 1-71 from the urine of human kidney transplant subjects who developed BKVIN (N=23) and kidney transplant patients who had a stable graft with no rejection or infection events (N=16, controls). Using NMR spectroscopy levels of metabolite resonances at 7.62+/−0.25 ppm×131.1+/−0.45 ppm, 3.12+/−0.25 ppm×32.81+/−0.45 ppm, 7.534+/−0.25 ppm×131.3+/−0.45 ppm, 7.82+/−0.25 ppm×129.7+/−0.45 ppm, 7.619+/−0.25 ppm×134.8+/−0.45 ppm, 3.97+/−0.25 ppm×46.51+/−0.45 ppm, 4.45+/−0.25 ppm×50.9+/−0.45 ppm, 2.19+/−0.25 ppm×24.54+/−0.45 ppm, 3.39+/−0.25 ppm×76.25+/−0.45 ppm, 2.89+/−0.25 ppm×32.96+/−0.45 ppm, 2.22+/−0.25 ppm×39.75+/−0.45 ppm, 3.75+/−0.25 ppm×62+/−0.45 ppm, 2.82+/−0.25 ppm×30+/−0.45 ppm, 3.38+/−0.25 ppm×76.2+/−0.45 ppm, 3.62+/−0.25 ppm×78.2+/−0.45 ppm, 4.05+/−0.25 ppm×58.5+/−0.45 ppm in the ¹H and ¹³C dimensions respectively were significantly different between BKVIN and control subjects (See FIG. 9 consisting of panels 9A-9P).

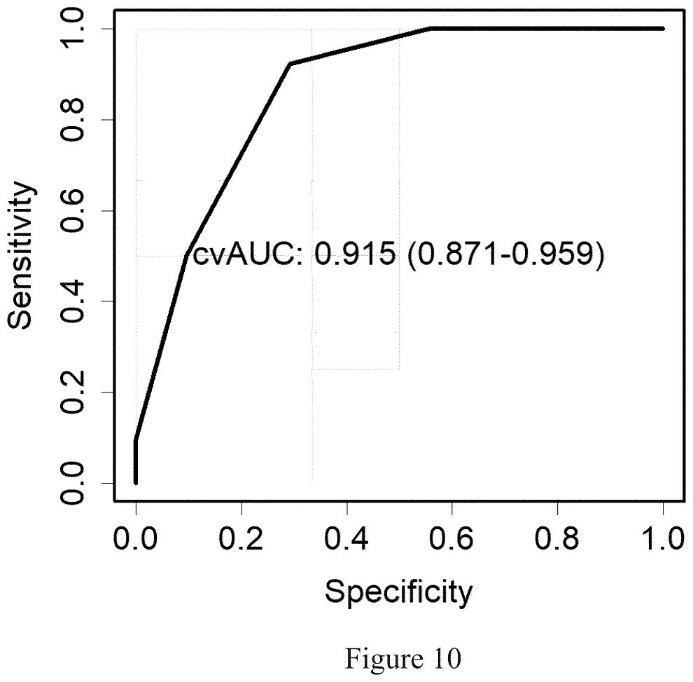

Using the differential levels of the above-mentioned metabolites, a machine learning algorithm produced a biomarker of response (BoR) score that differentiates kidney transplant subjects that develop BKVIN from controls with 91.5% cross-validated AUC (cvAUC) (see FIG. 10).

These results showed that metabolite biomarkers in urine are useful for identifying kidney transplant patients at risk for developing BKVIN. These results further indicated that methods and biomarkers of the present invention are useful for diagnosing BKVIN and other diseases or disorders associated with altered immune system response.

### Example 303-327

Metabolite Urine Levels in Human Male Kidney Transplant Subjects with Altered Immune System Response (ISR) Due to Over-Immunosuppression.

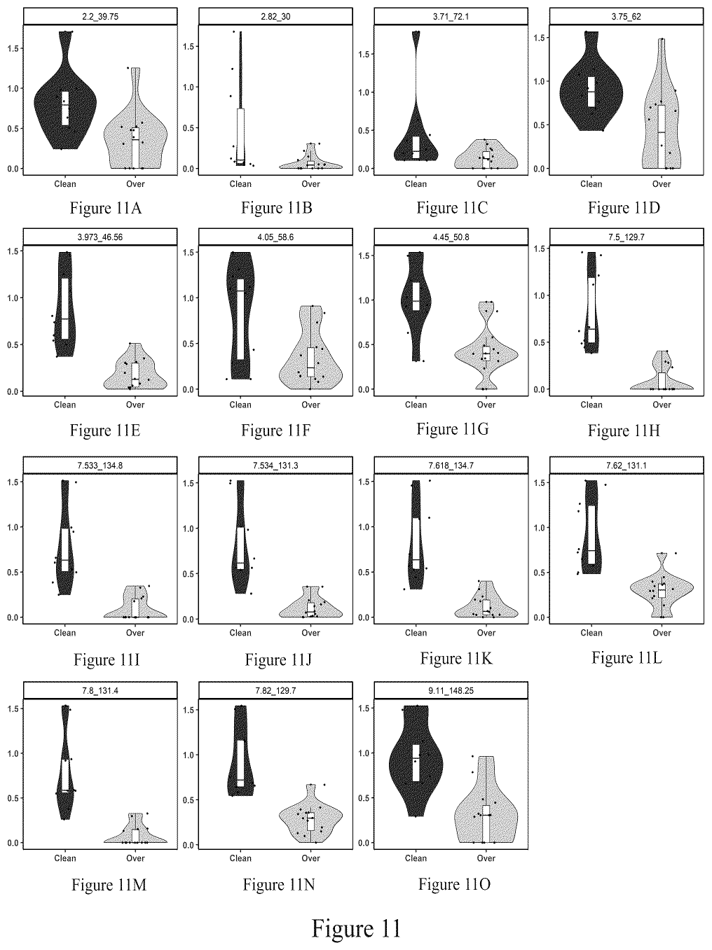

Levels of urine metabolites were assayed as described in previous examples from human male kidney transplant subjects who were over-immunosuppressed and with biopsy confirmed BKVIN (N=14) and compared to male kidney transplant patients who had a stable graft for over 2 years with no infection or rejection events (N=10, controls. Using NMR spectroscopy levels of metabolite resonances at 7.5+/−0.25 ppm×129.7+/−0.45 ppm, 7.533+/−0.25 ppm×134.8+/−0.45 ppm, 7.8+/−0.25 ppm×131.4+/−0.45 ppm, 7.618+/−0.25 ppm×134.7+/−0.45 ppm, 3.973+/−0.25 ppm×46.56+/−0.45 ppm, 7.534+/−0.25 ppm×131.3+/−0.45 ppm, 7.62+/−0.25 ppm×131.1+/−0.45 ppm, 7.82+/−0.25 ppm×129.7+/−0.45 ppm, 4.45+/−0.25 ppm×50.8+/−0.45 ppm, 9.11+/−0.25 ppm×148.25+/−0.45 ppm, 2.2+/−0.25 ppm×39.75+/−0.45 ppm, 3.75+/−0.25 ppm×62+/−0.45 ppm, 2.82+/−0.25 ppm×30+/−0.45 ppm, 4.05+/−0.25 ppm×58.6+/−0.45 ppm, 3.71+/−0.25 ppm×72.1+/−0.45 ppm in the ¹H and ¹³C dimensions respectively were significantly different between male over-immunosuppressed subjects and male control subjects (See FIG. 11 consisting of panels 11A-11O).

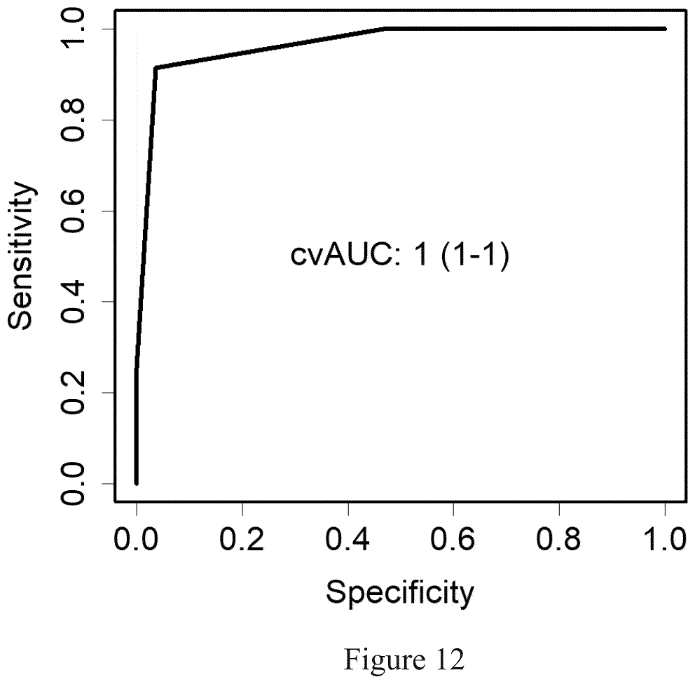

Using the differential levels of the above-mentioned metabolites, a machine learning algorithm produced a biomarker of response (BoR) score that differentiates that differentiates male kidney transplant subjects with biopsy confirmed BKVIN with 100% cvAUC (see FIG. 12).

These results showed that metabolite biomarkers in urine are useful for identifying male kidney transplant subjects with or at risk for complications due to over-immunosuppression. These results further indicated that methods and biomarkers of the present invention are useful for diagnosing over-immunosuppression and other diseases or disorders in male subjects associated with altered immune system response.

### Example 328-343

Metabolite Urine Levels in Human Female Kidney Transplant Subjects with Altered Immune System Response (ISR) Due to Over-Immunosuppression.

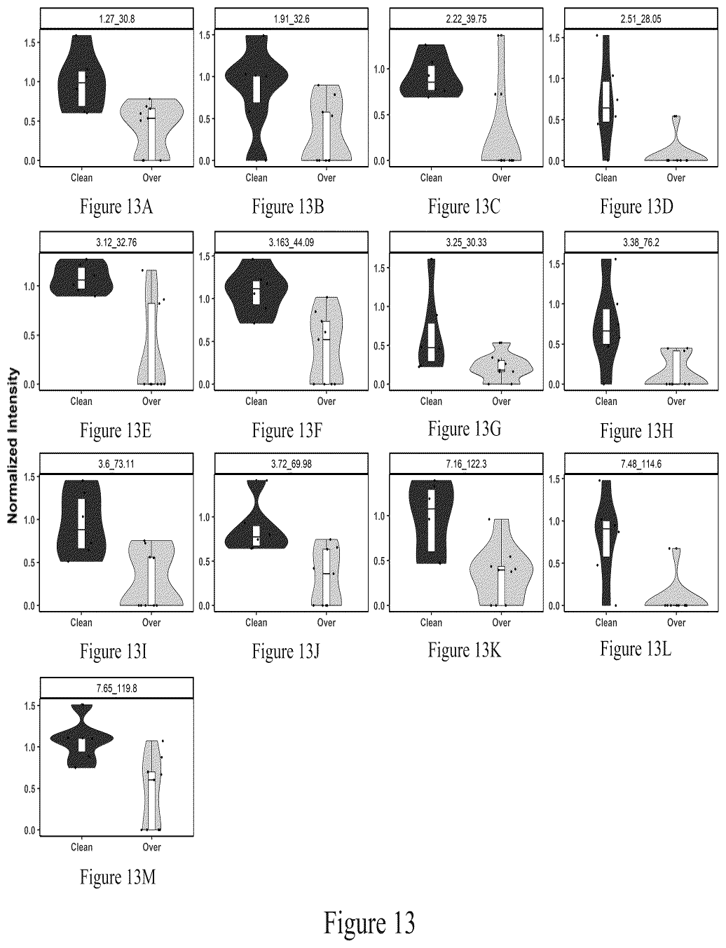

Levels of metabolites were assayed as described in previous examples from female human kidney transplant subjects who were over-immunosuppressed with biopsy confirmed BKVIN (N=9) and female kidney transplant patients who had a stable graft for over 2 years (N=6, controls). Using NMR spectroscopy levels of metabolite resonances at 7.16+/−0.25 ppm×122.3+/−0.45 ppm, 3.163+/−0.25 ppm×44.09+/−0.45 ppm, 1.27+/−0.25 ppm×30.8+/−0.45 ppm, 3.38+/−0.25 ppm×76.2+/−0.45 ppm, 3.12+/−0.25 ppm×32.76+/−0.45 ppm, 3.72+/−0.25 ppm×69.98+/−0.45 ppm, 2.51+/−0.25 ppm×28.05+/−0.45 ppm, 7.65+/−0.25 ppm×119.8+/−0.45 ppm, 2.22+/−0.25 ppm×39.75+/−0.45 ppm, 7.48+/−0.25 ppm×114.6+/−0.45 ppm, 3.6+/−0.25 ppm×73.11+/−0.45 ppm, 1.91+/−0.25 ppm×32.6+/−0.45 ppm, 3.25+/−0.25 ppm×30.33+/−0.45 ppm in the ¹H and ¹³C dimensions respectively were significantly different between female over-immunosuppressed subjects and female control subjects (See FIG. 13 consisting of panels 13A-13M).

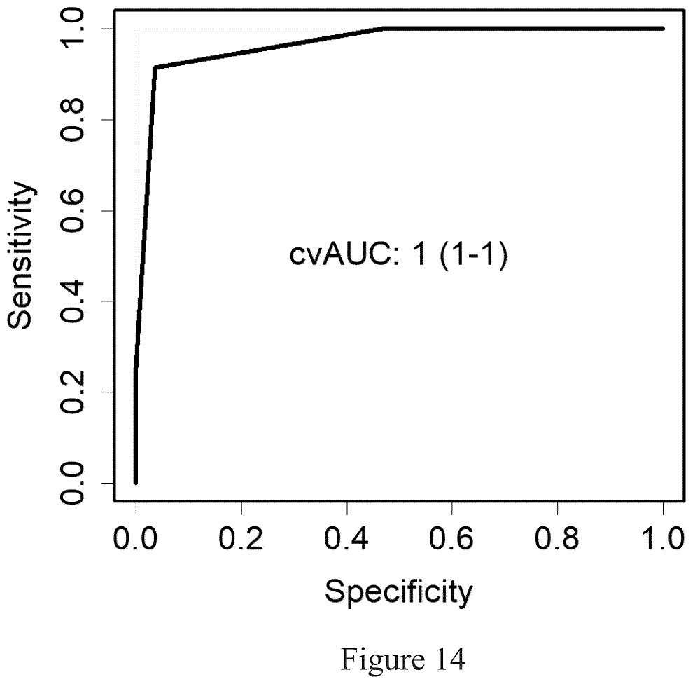

Using the differential levels of the above-mentioned metabolites, a machine learning algorithm produced a biomarker of response (BoR) score that differentiates that differentiates female kidney transplant subjects with biopsy confirmed BKVIN from controls with 100% cvAUC (see FIG. 14).

These results showed that metabolite biomarkers in urine are useful for identifying female kidney transplant subjects at risk for complications dues to over-immunosuppression. These results further indicated that methods and biomarkers of the present invention are useful for diagnosing over-immunosuppression and other diseases or disorders in female subjects associated with altered immune system response.

### REFERENCES

A, J. et al. “Extraction and GC/MS analysis of the human blood plasma metabolome.” Anal Chem 77, 8086-8094 (2005).
Buczko, Włodzimierz, Dorota Cylwik, and Wanda Stokowska. “Metabolism of tryptophan via the kynurenine pathway in saliva.” Postepy higieny i medycyny doswiadczalnej (Online) 59 (2005): 283-289.
Cacabelos, R., et al. “Therapeutic Effects of CDP-Choline in Alzheimer's Disease-Cognition, Brain Mapping, Cerebrovascular Hemodynamics, and Immune Factors a.” Annals of the New York Academy of Sciences 777.1 (1996): 399-403.
Chen, C., J. E. Sander, and N. M. Dale. “The effect of dietary lysine deficiency on the immune response to Newcastle disease vaccination in chickens.” Avian diseases 47.4 (2003): 1346-1351.
Chowdhury, Amrita A., et al. “Trigonelline insulates against oxidative stress, proinflammatory cytokines and restores BDNF levels in lipopolysaccharide induced cognitive impairment in adult mice.” Metabolic brain disease 33.3 (2018): 681-691.
Denburg, M. R. et al. “Metabolite Biomarkers of CKD Progression in Children”. Clin J Am Soc Nephrol 16, 1178-1189 (2021).
Diémé, B., Halimi, J. M., Emond, P., Büchler, M., Nadal-Desbarat, L., Blasco, H., & Le Guellec, C. “Assessing the metabolic effects of calcineurin inhibitors in renal transplant recipients by urine metabolic profiling”. Transplantation, 98 (2). (2014).\
Druml, W., Roth, E., Lenz, K., Lochs, H. & Kopsa, H. “Phenylalanine and tyrosine metabolism in renal failure: dipeptides as tyrosine source.” Kidney Int Suppl 27, S282-286 (1989).
Hall, C. J. & da Costa, T. P. S. Lysine: Biosynthesis, catabolismand roles. WikiJournal of Science (2018).
Haque, M. F., Aghabeighi, B., Wasil, M., Hodges, S. & Harris, M. “Oxygen free radicals in idiopathic facial pain.” Bangladesh Med Res Counc Bull 20, 104-116 (1994).
Human Metabolome Database (HMDB): Showing metabocard for D-Xylose (HMDB0000098). https://hmdb.ca/metabolites/HMDB0000098
Human Metabolome Database (HMDB): Showing metabocard for Pteroyltriglutamic acid (HMDB0001902). https://hmdb.ca/metabolites/HMDB0001902.
Human Metabolome Database (HMDB): Showing metabocard for Quinic acid (HMDB0003072). https://hmdb.ca/metabolites/HMDB0003072
Jawhar, S., Wirths, O. & Bayer, T. A. “Pyroglutamate amyloid-β (Aβ): a hatchet man in Alzheimer disease.” J Biol Chem 286, 38825-38832 (2011).
Ji, Yun, et al. “Anti-Inflammatory and Anti-Oxidative Activity of Indole-3-Acetic Acid Involves Induction of HO-1 and Neutralization of Free Radicals in RAW264. 7 Cells.” International Journal of Molecular Sciences 21.5 (2020): 1579.
Khanna-Gupta, Arati, and Nancy Berliner. “Vitamin B3 boosts neutrophil counts.” Nature medicine 15.2 (2009): 139-141.
Knoflach, Andreas, and U. Binswanger. “Serum hippuric acid concentration in renal allograft rejection, ureter obstruction, and tubular necrosis.” Transplant international 7.1 (1994): 17-21.
Kopple, J. D. “Phenylalanine and tyrosine metabolism in chronic kidney failure.” J Nutr 137, 1586S-1590S; discussion 1597S-1598S (2007).
Lee, R E. Nutritional Factors in Hemodynamics. AHA Journals (1952).
Mehta, Amit K., et al. “Choline attenuates immune inflammation and suppresses oxidative stress in patients with asthma.” Immunobiology 215.7 (2010): 527-534.
Moffett, John R., and MA ARYAN Namboodiri. “Tryptophan and the immune response.” Immunology and cell biology 81.4 (2003): 247-265.
Mullin, E. M., Bonar, R. A. & Paulson, D. F. “Acute tubular necrosis. An experimental model detailing the biochemical events accompanying renal injury and recovery.” Invest Urol 13, 289-294 (1976).
Palego, Lionella, et al. “Tryptophan biochemistry: structural, nutritional, metabolic, and medical aspects in humans.” Journal of Amino Acids 2016 (2016).
Pero, R. W. “Health Consequences of Catabolic Synthesis of Hippuric Acid in Humans.” Current Clinical Pharmacology 5, 67-73 (2010).
PubChem. Aminohippuric acid. https://pubchem.ncbi.nlm.nih.gov/compound/2148.
PubChem. Creatine. https://pubchem.ncbi.nlm.nih.gov/compound/586
PubChem. 3-Hydroxymandelic acid. https://pubchem.ncbi.nlm.nih.gov/compound/86957.
PubChem. 4-Methoxyphenylacetic acid. https://pubchem.ncbi.nlm.nih.gov/compound/7690.
PubChem. Phenylalanine. https://pubchem.ncbi.nlm.nih.gov/compound/6140.
PubChem. Salicyluric acid. https://pubchem.ncbi.nlm.nih.gov/compound/10253.
Saadati, Shaghayegh, et al. “A surface-enhanced Raman scattering-based approach for rapid and highly sensitive quantitative analysis of 3-carboxy-4-methyl-5-propyl-2-furanpropionate and indole-3-acetic acid in saline, human serum and uremic serum of patients with chronic kidney disease.” RSC Advances 10.71 (2020): 43489-43496.
Sadeghi, Mahmoud, et al. “Strong association of phenylalanine and tryptophan metabolites with activated cytomegalovirus infection in kidney transplant recipients.” Human immunology 73.2 (2012): 186-192.
Slominski, Andrzej, et al. “Conversion of L-tryptophan to serotonin and melatonin in human melanoma cells.” FEBS letters 511.1-3 (2002): 102-106.
Weinstein, Islas, Leon, Alberto Revuelta, and Rogelio Hernandez Pando. “Catecholamines and acetylcholine are key regulators of the interaction between microbes and the immune system.” Annals of the New York Academy of Sciences 1351.1 (2015): 39-51.
Wikoff, William R., et al. “Metabolomics analysis reveals large effects of gut microflora on mammalian blood metabolites.” Proceedings of the national academy of sciences 106.10 (2009)
Zeisel, Steven H. “Choline: an essential nutrient for humans.” Nutrition. 2000.

1. A method of analyzing an immune system response of a kidney transplant patient, the method comprising:
a. obtaining a biological sample from the kidney transplant patient;
b. using nuclear magnetic resonance (NMR) spectroscopy to detect metabolites in the biological sample related to the immune system response, wherein intensities of one or more metabolite resonances are used to classify the kidney transplant patient as under-immunosuppressed, over-immunosuppressed, or stable using a machine learning model,
wherein the intensities of one or more metabolite resonances for one or more metabolites having chemical shifts at 2.711 ¹H ppm and 47.702 ¹³C ppm, 3.969 ¹H ppm and 46.484 ¹³C ppm, 7.086 ¹H ppm and 121.698 ¹³C ppm, 7.274 ¹H ppm and 116.53 ¹³C ppm, 7.347 ¹H ppm and 121.578 ¹³C ppm, 7.532 ¹H ppm and 129.75 ¹³C ppm, 7.532 ¹H ppm and 131.359 ¹³C ppm, 7.533 ¹H ppm and 134.812 ¹³C ppm, 7.618 ¹H ppm and 134.804 ¹³C ppm, 7.817 ¹H ppm and 131.389 ¹³C ppm, 7.818 ¹H ppm and 129.746 ¹³C ppm, 9.117 ¹H ppm and 148.321 ¹³C ppm are used to calculate a score for kidney graft stability in the kidney transplant patient using the machine learning model, wherein the score is used to classify the kidney transplant patient as having appropriate immunosuppression or not having appropriate immunosuppression, and
wherein the intensities of one or more metabolite resonances for one or more metabolites having chemical shifts at 3.851 ¹H ppm and 64.395 ¹³C ppm, 7.534 ¹H ppm and 134.842 ¹³C ppm, 2.787 ¹H ppm and 40.035 ¹³C ppm, 6.914 ¹H ppm and 115.449 ¹³C ppm, 7.088 ¹H ppm and 121.707 ¹³C ppm, 3.714 ¹H ppm and 72.229 ¹³C ppm, 3.637 ¹H ppm and 78.911 ¹³C ppm, 6.974 ¹H ppm and 120.651 ¹³C ppm, 7.817 ¹H ppm and 131.398 ¹³C ppm, 7.534 ¹H ppm and 129.772 ¹³C ppm, 3.966 ¹H ppm and 46.482 ¹³C ppm, 3.01 ¹H ppm and 32.525 ¹³C ppm, 2.512 ¹H ppm and 28.032 ¹³C ppm, 7.533 ¹H ppm and 131.372 ¹³C ppm, 7.619 ¹H ppm and 134.811 ¹³C ppm, 3.722 ¹H ppm and 75.654 ¹³C ppm, 7.276 ¹H ppm and 116.555 ¹³C ppm, 5.016 ¹H ppm and 74.051 ¹³C ppm are used to further classify the kidney transplant patient as over-immunosuppressed or under-immunosuppressed using the machine learning model if the kidney transplant patient is classified as not having appropriate immunosuppression; and
c. increasing an immunosuppressant dose if the renal transplant recipient is classified as under-immunosuppressed, or decreasing the immunosuppressant dose if the renal transplant recipient is classified as over-immunosuppressed.

2. The method of claim 1, wherein the intensities of one or more metabolite resonances for one or more metabolites having chemical shifts at 2.792 ¹H ppm and 40.011 ¹³C ppm, 3.714 ¹H ppm and 72.206 ¹³C ppm, and 3.009 ¹H ppm and 32.551 ¹³C ppm are used to further classify the kidney transplant patient as under-immunosuppressed or stable using the machine learning model.

3. The method of claim 1, wherein the intensities of one or more metabolite resonances for one or more metabolites having chemical shifts at 7.62 ¹H ppm and 131.1 ¹³C ppm, 3.12 ¹H ppm and 32.81 ¹³C ppm, 7.534 ¹H ppm and 131.3 ¹³C ppm, 7.82 ¹H ppm and 129.7 ¹³C ppm, 7.619 ¹H ppm and 134.8 ¹³C ppm, 3.97 ¹H ppm and 46.51 ¹³C ppm, 4.45 ¹H ppm and 50.9 ¹³C ppm, 2.19 ¹H ppm and 24.54 ¹³C ppm, 3.39 ¹H ppm and 76.25 ¹³C ppm, 2.89 ¹H ppm and 32.96 ¹³C ppm, 2.22 ¹H ppm and 39.75 ¹³C ppm, 3.75 ¹H ppm and 62 ¹³C ppm, 2.82 ¹H ppm and 30 ¹³C ppm, 3.38 ¹H ppm and 76.2 ¹³C ppm, 3.62 ¹H ppm and 78.2 ¹³C ppm, and 4.05 ¹H ppm and 58.5 ¹³C ppm are used to further classify the kidney transplant patient as over-immunosuppressed or stable using the machine learning model.

4. The method of claim 1, wherein the biological sample is a urine sample.

5. The method of claim 1, wherein the kidney transplant patient is diagnosed with BK-Virus or BK Virus Interstitial Nephritis (BKVIN).

6. The method of claim 1, wherein the kidney transplant patient is diagnosed with graft dysfunction, transplant rejection, or organ failure.

7. The method of claim 1, wherein the kidney transplant patient is diagnosed with complications associated with overimmunosuppression such as infection, post-transplant diabetes, malignancy, or nephrotoxicity.

8. The method of claim 2, wherein an increase in the intensity of the metabolite resonance for the metabolite having chemical shifts at 2.792 ¹H ppm and 40.011 ¹³C ppm, and a decrease in the intensities of the metabolite resonances for the metabolites having chemical shifts at 3.714 ¹H ppm and 72.206 ¹³C ppm, and 3.009 ¹H ppm and 32.551 ¹³C ppm for the kidney transplant patient compared to a control subject indicate that the kidney transplant patient is under-immunosuppressed.

9. The method of claim 1, wherein an increase in the intensities of the metabolite resonances for the metabolites having chemical shifts at 2.512 ¹H ppm and 28.032 ¹³C ppm, 2.787 ¹H ppm and 40.035 ¹³C ppm, 3.966 ¹H ppm and 46.482 ¹³C ppm, 5.016 ¹H ppm and 74.051 ¹³C ppm, 6.914 ¹H ppm and 115.449 ¹³C ppm, 6.974 ¹H ppm and 120.651 ¹³C ppm, 7.088 ¹H ppm and 121.707 ¹³C ppm, 7.276 ¹H ppm and 116.555 ¹³C ppm, 7.533 ¹H ppm and 131.372 ¹³C ppm, 7.534 ¹H ppm and 129.772 ¹³C ppm, 7.534 ¹H ppm and 134.842 ¹³C ppm, 7.619 ¹H ppm and 134.811 ¹³C ppm, and 7.817 ¹H ppm and 131.398 ¹³C ppm; and a decrease in the intensities of the metabolite resonances for the metabolites having chemical shifts at 3.01 ¹H ppm and 32.525 ¹³C ppm, 3.637 ¹H ppm and 78.911 ¹³C ppm, 3.714 ¹H ppm and 72.229 ¹³C ppm, 3.722 ¹H ppm and 75.654 ¹³C ppm, and 3.851 ¹H ppm and 64.395 ¹³C ppm for the kidney transplant patient compared to an over-immunosuppressed subject indicate that the kidney transplant patient is under-immunosuppressed.

10. The method of claim 1, wherein a decrease in the intensities of the metabolite resonances for the metabolites having chemical shifts at 2.512 ¹H ppm and 28.032 ¹³C ppm, 2.787 ¹H ppm and 40.035 ¹³C ppm, 3.966 ¹H ppm and 46.482 ¹³C ppm, 5.016 ¹H ppm and 74.051 ¹³C ppm, 6.914 ¹H ppm and 115.449 ¹³C ppm, 6.974 ¹H ppm and 120.651 ¹³C ppm, 7.088 ¹H ppm and 121.707 ¹³C ppm, 7.276 ¹H ppm and 116.555 ¹³C ppm, 7.533 ¹H ppm and 131.372 ¹³C ppm, 7.534 ¹H ppm and 129.772 ¹³C ppm, 7.534 ¹H ppm and 134.842 ¹³C ppm, 7.619 ¹H ppm and 134.811 ¹³C ppm, and 7.817 ¹H ppm and 131.398 ¹³C ppm; and an increase in the intensities of the metabolite resonances for the metabolites having chemical shifts at 3.01 ¹H ppm and 32.525 ¹³C ppm, 3.637 ¹H ppm and 78.911 ¹³C ppm, 3.714 ¹H ppm and 72.229 ¹³C ppm, 3.722 ¹H ppm and 75.654 ¹³C ppm, and 3.851 ¹H ppm and 64.395 ¹³C ppm for the kidney transplant patient compared to an under-immunosuppressed subject indicate that the kidney transplant patient is over-immunosuppressed.

11. The method of claim 1, wherein an increase in the intensity of the metabolite resonance for the metabolite having chemical shifts at 2.711 ¹H ppm and 47.702 ¹³C ppm; and a decrease in the intensities of the metabolite resonances for the metabolites having chemical shifts at 3.969 ¹H ppm and 46.484 ¹³C ppm, 7.086 ¹H ppm and 121.698 ¹³C ppm, 7.274 ¹H ppm and 116.53 ¹³C ppm, 7.347 ¹H ppm and 121.578 ¹³C ppm, 7.532 ¹H ppm and 129.75 ¹³C ppm, 7.532 ¹H ppm and 131.359 ¹³C ppm, 7.533 ¹H ppm and 134.812 ¹³C ppm, 7.618 ¹H ppm and 134.804 ¹³C ppm, 7.817 ¹H ppm and 131.389 ¹³C ppm, 7.818 ¹H ppm and 129.746 ¹³C ppm, and 9.117 ¹H ppm and 148.321 ¹³C ppm for the kidney transplant patient compared to a stable subject indicate that the kidney transplant patient does not have appropriate immunosuppression.

12. The method of claim 3, wherein a decrease in the intensities of the metabolite resonances for the metabolites having chemical shifts at 2.19 ¹H ppm and 24.54 ¹³C ppm, 2.22 ¹H ppm and 39.75 ¹³C ppm, 2.82 ¹H ppm and 30 ¹³C ppm, 2.89 ¹H ppm and 32.96 ¹³C ppm, 3.12 ¹H ppm and 32.81 ¹³C ppm, 3.38 ¹H ppm and 76.2 ¹³C ppm, 3.39 ¹H ppm and 76.25 ¹³C ppm, 3.62 ¹H ppm and 78.2 ¹³C ppm, 3.75 ¹H ppm and 62 ¹³C ppm, 3.97 ¹H ppm and 46.51 ¹³C ppm, 4.05 ¹H ppm and 58.5 ¹³C ppm, 4.45 ¹H ppm and 50.9 ¹³C ppm, 7.534 ¹H ppm and 131.3 ¹³C ppm, 7.619 ¹H ppm and 134.8 ¹³C ppm, 7.62 ¹H ppm and 131.1 ¹³C ppm, and 7.82 ¹H ppm and 129.7 ¹³C ppm for the kidney transplant patient compared to a control subject indicate that the kidney transplant patient is over-immunosuppressed.

13. The method of claim 1, further comprising using the intensities of one or more metabolite resonances for one or more metabolites having chemical shifts at 7.5 ¹H ppm and 129.7 ¹³C ppm, 7.533 ¹H ppm and 134.8 ¹³C ppm, 7.8 ¹H ppm and 131.4 ¹³C ppm, 7.618 ¹H ppm and 134.7 ¹³C ppm, 3.973 ¹H ppm and 46.56 ¹³C ppm, 7.534 ¹H ppm and 131.3 ¹³C ppm, 7.62 ¹H ppm and 131.1 ¹³C ppm, 7.82 ¹H ppm and 129.7 ¹³C ppm, 4.45 ¹H ppm and 50.8 ¹³C ppm, 9.11 ¹H ppm and 148.25 ¹³C ppm, 2.2 ¹H ppm and 39.75 ¹³C ppm, 3.75 ¹H ppm and 62 ¹³C ppm, 2.82 ¹H ppm and 30 ¹³C ppm, 4.05 ¹H ppm and 58.6 ¹³C ppm, 3.71 ¹H ppm and 72.1 ¹³C ppm to classify the kidney transplant patient as over-immunosuppressed or stable using the machine learning model if the kidney transplant patient is male.

14. The method of claim 13, wherein a decrease in the intensities of the metabolite resonances for the metabolites having chemical shifts at 7.5 ¹H ppm and 129.7 ¹³C ppm, 7.533 ¹H ppm and 134.8 ¹³C ppm, 7.8 ¹H ppm and 131.4 ¹³C ppm, 7.618 ¹H ppm and 134.7 ¹³C ppm, 3.973 ¹H ppm and 46.56 ¹³C ppm, 7.534 ¹H ppm and 131.3 ¹³C ppm, 7.62 ¹H ppm and 131.1 ¹³C ppm, 7.82 ¹H ppm and 129.7 ¹³C ppm, 4.45 ¹H ppm and 50.8 ¹³C ppm, 9.11 ¹H ppm and 148.25 ¹³C ppm, 2.2 ¹H ppm and 39.75 ¹³C ppm, 3.75 ¹H ppm and 62 ¹³C ppm, 2.82 ¹H ppm and 30 ¹³C ppm, 4.05 ¹H ppm and 58.6 ¹³C ppm, 3.71 ¹H ppm and 72.1 ¹³C ppm for the male kidney transplant patient compared to a control subject indicate that the male kidney transplant patient is over-immunosuppressed.

15. The method of claim 1, further comprising using the intensities of one or more metabolite resonances for one or more metabolites having chemical shifts at 7.16 ¹H ppm and 122.3 ¹³C ppm, 3.163 ¹H ppm and 44.09 ¹³C ppm, 1.27 ¹H ppm and 30.8 ¹³C ppm, 3.38 ¹H ppm and 76.2 ¹³C ppm, 3.12 ¹H ppm and 32.76 ¹³C ppm, 3.72 ¹H ppm and 69.98 ¹³C ppm, 2.51 ¹H ppm and 28.05 ¹³C ppm, 7.65 ¹H ppm and 119.8 ¹³C ppm, 2.22 ¹H ppm and 39.75 ¹³C ppm, 7.48 ¹H ppm and 114.6 ¹³C ppm, 3.6 ¹H ppm and 73.11 ¹³C ppm, 1.91 ¹H ppm and 32.6 ¹³C ppm, 3.25 ¹H ppm and 30.33 ¹³C ppm to classify the kidney transplant patient as over-immunosuppressed or stable using the machine learning model if the kidney transplant patient is female.

16. The method of claim 15, wherein a decrease in the intensities of the metabolite resonances for the metabolites having chemical shifts at 7.16 ¹H ppm and 122.3 ¹³C ppm, 3.163 ¹H ppm and 44.09 ¹³C ppm, 1.27 ¹H ppm and 30.8 ¹³C ppm, 3.38 ¹H ppm and 76.2 ¹³C ppm, 3.12 ¹H ppm and 32.76 ¹³C ppm, 3.72 ¹H ppm and 69.98 ¹³C ppm, 2.51 ¹H ppm and 28.05 ¹³C ppm, 7.65 ¹H ppm and 119.8 ¹³C ppm, 2.22 ¹H ppm and 39.75 ¹³C ppm, 7.48 ¹H ppm and 114.6 ¹³C ppm, 3.6 ¹H ppm and 73.11 ¹³C ppm, 1.91 ¹H ppm and 32.6 ¹³C ppm, 3.25 ¹H ppm and 30.33 ¹³C ppm for the female kidney transplant patient compared to a control subject indicate that the female kidney transplant patient is over-immunosuppressed.

17. The method of claim 1, wherein the one or more metabolites are selected from choline, hippuric acid, indole-3-acetic acid, lysine, trigonelline, tryptophan, 2-pyrocatechuic-acid, 3-hydroxymandelic acid, 1-phenylalanine,4-methoxyphenylacetic acid, 4-aminohippuric acid, pteroyltriglutamic acid, 4-ethylbenzoic acid, aspartylphenylalanine, creatinine, diphenhydramine, D-xylose, gulonic acid, homoveratric acid, pyroglutamic acid, quinic acid, and salicyluric acid.

18. The method of claim 1, wherein the NMR spectroscopy is heteronuclear single-quantum correlation (HSQC) two-dimensional NMR spectroscopy.

19. The method of claim 1, wherein the intensities of the metabolite resonances for the metabolites having chemical shifts at 2.711 ¹H ppm and 47.702 ¹³C ppm, 3.969 ¹H ppm and 46.484 ¹³C ppm, 7.086 ¹H ppm and 121.698 ¹³C ppm, 7.274 ¹H ppm and 116.53 ¹³C ppm, 7.347 ¹H ppm and 121.578 ¹³C ppm, 7.532 ¹H ppm and 129.75 ¹³C ppm, 7.532 ¹H ppm and 131.359 ¹³C ppm, 7.533 ¹H ppm and 134.812 ¹³C ppm, 7.618 ¹H ppm and 134.804 ¹³C ppm, 7.817 ¹H ppm and 131.389 ¹³C ppm, 7.818 ¹H ppm and 129.746 ¹³C ppm, 9.117 ¹H ppm and 148.321 ¹³C ppm are used to calculate a score for kidney graft stability in the kidney transplant patient.

20. The method of claim 19, wherein the intensities of the metabolite resonances for the metabolites having chemical shifts at 3.851 ¹H ppm and 64.395 ¹³C ppm, 7.534 ¹H ppm and 134.842 ¹³C ppm, 2.787 ¹H ppm and 40.035 ¹³C ppm, 6.914 ¹H ppm and 115.449 ¹³C ppm, 7.088 ¹H ppm and 121.707 ¹³C ppm, 3.714 ¹H ppm and 72.229 ¹³C ppm, 3.637 ¹H ppm and 78.911 ¹³C ppm, 6.974 ¹H ppm and 120.651 ¹³C ppm, 7.817 ¹H ppm and 131.398 ¹³C ppm, 7.534 ¹H ppm and 129.772 ¹³C ppm, 3.966 ¹H ppm and 46.482 ¹³C ppm, 3.01 ¹H ppm and 32.525 ¹³C ppm, 2.512 ¹H ppm and 28.032 ¹³C ppm, 7.533 ¹H ppm and 131.372 ¹³C ppm, 7.619 ¹H ppm and 134.811 ¹³C ppm, 3.722 ¹H ppm and 75.654 ¹³C ppm, 7.276 ¹H ppm and 116.555 ¹³C ppm, 5.016 ¹H ppm and 74.051 ¹³C ppm are used to further classify the kidney transplant patient as over-immunosuppressed or under-immunosuppressed.
